SEQUENCE LISTING (16 sequences)

SEQ ID NO: 1 - DNA, 20 bp, Artificial Sequence
  source: /note=“Description of Artificial Sequence Synthetic primer”
tgcaccaccaactgcttagc

SEQ ID NO: 2 - DNA, 21 bp, Artificial Sequence
  source: /note=“Description of Artificial Sequence Synthetic primer”
ggcatggactgtggtcatgag

SEQ ID NO: 3 - DNA, 20 bp, Artificial Sequence
  source: /note=“Description of Artificial Sequence Synthetic primer”
ggagccatggattgcacttt

SEQ ID NO: 4 - DNA, 21 bp, Artificial Sequence
  source: /note=“Description of Artificial Sequence Synthetic primer”
tcaaataggccagggaagtca

SEQ ID NO: 5 - DNA, 21 bp, Artificial Sequence
  source: /note=“Description of Artificial Sequence Synthetic primer”
cataattcccgacgtggcttt

SEQ ID NO: 6 - DNA, 26 bp, Artificial Sequence
  source: /note=“Description of Artificial Sequence Synthetic primer”
aggtttgtagtacctcctctggaaca

SEQ ID NO: 7 - DNA, 18 bp, Artificial Sequence
  source: /note=“Description of Artificial Sequence Synthetic primer”
gaccgcttccgagattcc

SEQ ID NO: 8 - DNA, 18 bp, Artificial Sequence
  source: /note=“Description of Artificial Sequence Synthetic primer”
gatggcagtcaggctcac

SEQ ID NO: 9 - DNA, 22 bp, Artificial Sequence
  source: /note=“Description of Artificial Sequence Synthetic primer”
tgtctacagaaaacccaagtgg

SEQ ID NO: 10 - DNA, 21 bp, Artificial Sequence
  source: /note=“Description of Artificial Sequence Synthetic primer”
tgtggaagatgttaggcttgg

SEQ ID NO: 11 - DNA, 20 bp, Artificial Sequence
  source: /note=“Description of Artificial Sequence Synthetic primer”
gccaagctgctccagaaaat

SEQ ID NO: 12 - DNA, 21 bp, Artificial Sequence
  source: /note=“Description of Artificial Sequence Synthetic primer”
tgatcacctgcagtagctgca

SEQ ID NO: 13 - DNA, 22 bp, Artificial Sequence
  source: /note=“Description of Artificial Sequence Synthetic primer”
agccactcacctcttcagaacg

SEQ ID NO: 14 - DNA, 22 bp, Artificial Sequence
  source: /note=“Description of Artificial Sequence Synthetic primer”
ggttcaggttgttttctgccag

SEQ ID NO: 15 - DNA, 21 bp, Artificial Sequence
  source: /note=“Description of Artificial Sequence Synthetic primer”
tctacaagcccatgcttcgac

SEQ ID NO: 16 - DNA, 18 bp, Artificial Sequence
  source: /note=“Description of Artificial Sequence Synthetic primer”
ggacgctcaccaggtact

(19) United States
(12) Patent Application Publication
(10) Pub. No.: US 2022/0000818 A1
(43) Pub. Date: Jan. 6, 2022

(54) COMPOSITIONS FOR USE IN PREVENTING ACNE

(71) Applicant: Nogra Pharma Limited, Dublin (IE)

(72) Inventors: Salvatore Bellinvia, Mendrisio (CH); Francesca Viti, Salorino (CH)

(21) Appl. No.: 17/425,648

(22) PCT Filed: Jan. 24, 2020
(86) PCT No.: PCT/EP2020/051810

§ 371 Date: Jul. 23, 2021

Related U.S. Application Data
(60) Provisional application No. 62/796,795, filed on Jan. 25, 2019.

(51) Int. Cl. A61K 31/196; A61P 17/10; A61K 9/06

(52) U.S. Cl. CPC A61K 31/196; A61K 9/0014; A61K 9/06; A61P 17/10

(57) ABSTRACT

Disclosed herein are methods for preventing acne in a patient comprising administering topically to the patient a composition effective to induce sebocyte differentiation. Also disclosed herein are methods of preventing acne in a patient comprising administering topically to the patient a composition effective to reduce insulin-induced lipoxygenase (LOX) activity and/or inflammatory processes. Also disclosed herein are methods of preventing a disease, condition, or disorder characterized by alteration of sebocyte differentiation, comprising administering topically to a patient a composition effective to induce sebocyte differentiation. The described methods can include administering a composition that includes a therapeutically effective amount of a PPARγ modulator, and a pharmaceutically acceptable excipient.

This application claims the benefit of and priority to U.S. Provisional Patent Application No. 62/796,795, filed Jan. 25, 2019, the entire contents of which are incorporated by reference herein for all purposes.

### SEQUENCE LISTING

The instant application contains a Sequence Listing which has been submitted electronically in ASCII format and is hereby incorporated by reference in its entirety. Said ASCII copy, created on Jan. 23, 2020, is named P133964WO_SL.txt and is 4,195 bytes in size.

### BACKGROUND

Acne is the most common skin disorder globally and in the United States of America, affecting 40 to 50 million Americans each year. In 2015, acne was estimated to affect 633 million people globally or almost 10% of the global population, making it the eighth most common disease worldwide. Generally, the prevalence of acne continues to grow globally in all regions except Sub-Saharan Africa, although a higher disease prevalence and rate of growth is observed in wealthier regions such as Western Europe, high-income Asian Pacific regions, the United States of America, and Canada.

Acne commonly occurs in adolescence and affects an estimated 80-90% of teenagers in the Western world, including 85% of individuals between the ages of 12 and 25. Children and adults may also be affected before and after puberty. Although acne becomes less common in adulthood, it persists in nearly half of affected people into their twenties (64% of individuals) and thirties (43% of individuals) and a smaller group continue to have difficulties into their forties. Acne also has a significant public health cost. For instance, in the United States of America over $3 billion dollars per year is lost to the cost of treatment and loss of productivity.

Acne is a multifactorial pathology of the sebaceous gland, characterized by the presence of a number of physical skin features, including blackheads, whiteheads, papules, pustules (also known as pimples), cysts, and nodules. Acne can result in permanent dark spots and scars if not treated.

Aside from its physical effects, acne can have severe psychological, social, and emotional effects, especially in teenagers. For instance, acne can cause decreased self-esteem and confidence, resulting in decreased social interaction and decreased work and school attendance (with negative effects on employment and academic performance, respectively). Distress caused by acne can also result in depression, as well as suicidal ideation in individuals suffering from severe acne.

In general, acne results from alterations in oil production, clogging of hair follicles sebum and dead skin cells, bacterial growth, and/or increased androgen levels. Acne development also appears to be linked, in part, to the Western diet, which includes high levels of hyperglycemic carbohydrates and insulinotropic dairy products. Notably, insulin/IGF-1 signaling are also reported to play a role in inducing sebogenesis and inflammation. Stress also appears to worsen symptoms of acne. There also appears to be a significant genetic component to acne.

Given the prevalence of acne, there is a need for development of robust methods for preventing acne and other related diseases, conditions, and disorders.

### SUMMARY

It has now been discovered that acne can be prevented by administering a therapeutically effective amount of a Peroxisome Proliferator-Activated Receptor Gamma (PPARγ) modulator. More specifically, described herein are methods of preventing acne in a patient, comprising administering topically to the patient a composition effective to induce sebocyte differentiation, where the composition comprises a therapeutically effective amount of a PPARγ modulator, and a pharmaceutically acceptable excipient. Also described herein are methods of preventing acne in a patient, comprising administering topically to the patient a composition effective to reduce insulin-induced lipoxygenase (LOX) activity, where the composition comprises a therapeutically-effective amount of a PPARγ modulator, and a pharmaceutically acceptable excipient. In some embodiments, the patient displays no signs or symptoms of acne.

Also provided herein are methods of preventing acne and/or a disease, a condition, or a disorder characterized by alteration of sebocyte differentiation in a patient, comprising administering topically to the patient a composition effective to induce sebocyte differentiation, wherein the composition comprises a therapeutically effective amount of a PPARγ modulator, and a pharmaceutically acceptable excipient. In some embodiments, the disease, the condition, or the disorder is selected from one or more of acne, sebaceous hyperplasia, and sebaceous adenitis.

In some embodiments, the methods described herein restore the physiological composition of secreted sebum in a patient with acne to a physiological composition as found in patients (or a patient) without acne. For example, in some embodiments, the methods restore the physiological composition of secreted sebum (for example, the methods restore the physiological composition of secreted sebum in a patient with acne) to a level of mono-unsaturated fatty acids as found in patients (or a patient) without acne. For example, the described methods can restore the physiological composition of secreted sebum in a patient with acne to a level of mono-unsaturated acids, for example, C16:1 mono-unsaturated fatty acids as found in patients (or a patient) without acne. The described methods can restore a level of a mono-unsaturated acid of secreted sebum in a patient with acne to a level of the mono-unsaturated acid, for example, a C16:1 mono-unsaturated fatty acid such as palmitoleic acid and/or sapienic acid, as found in patients (or a patient) without acne. In some embodiments, the methods restore the physiological composition of secreted sebum (for example, the methods restore the physiological composition of secreted sebum in a patient with acne) to a level of diacylglycerides as found in patients (or a patient) without acne. For example, the described methods can restore a level of a diacylglyceride of secreted sebum in a patient with acne to a level of the diacylglyceride as found in patients (or a patient) without acne.

In some embodiments, the PPARγ modulator is a class of compounds including N-acetyl-3-(4′-aminophenyl)-2-methoxypropionic acid. In certain embodiments, the PPARγ modulator is N-acetyl-3-(4′-aminophenyl)-2-methoxypropionic acid, or a stereoisomer and/or a pharmaceutically acceptable salt thereof. For example, in certain embodiments, the PPARγ modulator is N-acetyl-(S)-3-(4′-aminophenyl)-2-methoxypropionic acid (“NAC-GED0507”), or a pharmaceutically acceptable salt thereof. In certain embodiments, the PPARγ modulator is N-acetyl-(R)-3-(4′-aminophenyl)-2-methoxypropionic acid, or a pharmaceutically acceptable salt thereof. In certain embodiments, the PPARγ modulator is a racemic mixture of N-acetyl-(R)-3-(4′-aminophenyl)-2-methoxypropionic acid and N-acetyl-(S)-3-(4′-aminophenyl)-2-methoxypropionic acid, or one or more pharmaceutically acceptable salts thereof. In some embodiments, the PPARγ modulator is a mixture of the (S) and (R) enantiomers of N-acetyl-3-(4′-aminophenyl)-2-methoxypropionic acid, where the ratio of (S):(R) enantiomers is between <100:>0 and >0:<100, which includes a racemic mixture (50:50).

In certain embodiments, the patient to whom a composition is administered in one of the methods described herein possesses certain characteristics or meets certain criteria. For example, in some embodiments, the patient is experiencing puberty. In certain embodiments, the patient has eaten or is eating an insulinotropic diet (for example, a Western diet).

In certain embodiments, compositions used in one or more of the methods described herein may be administered by various means, depending on their intended use, as is well known in the art. For example, compositions disclosed herein may be administered by one or several routes, including topically, parenterally (e.g., subcutaneously, intramuscularly, intradermally, or intravenously), orally, buccally, rectally, pulmonarily, intratracheally, intranasally, transdermally, or intraduodenally. Compositions of the present invention may be administered topically, for example, in the form of a cream, a gel, an ointment, a wax, a powder, a liquid, a liquid spray, or a foam such as an aerosol foam. If compositions of the present invention are to be administered orally, they may be formulated as tablets, capsules, granules, powders, or syrups. Alternatively, compositions of the present invention may be administered parenterally as injections (for example, intravenous, intramuscular, or subcutaneous), drop infusion preparations, enemas, or suppositories.

Also described herein are compositions effective to induce sebocyte differentiation for use in methods of preventing acne in a patient, comprising administering topically to the patient the composition, wherein the composition comprises a PPARγ modulator, and a pharmaceutically acceptable excipient. Also described herein are compositions effective to reduce insulin-induced lipoxygenase (LOX) activity for use in methods of preventing acne in a patient, comprising administering topically to the patient the composition, wherein the composition comprises a PPARγ modulator, and a pharmaceutically acceptable excipient. In some embodiments, the patient displays no signs or symptoms of acne. Any features described and/or claimed in relation to a method described herein apply mutatis mutandis to said compositions for use.

Also described herein are compositions effective to induce sebocyte differentiation for use in methods of preventing acne and/or a disease, a condition, or a disorder characterized by alteration of sebocyte differentiation in a patient, comprising administering topically to the patient the composition, wherein the composition comprises a PPARγ modulator, and a pharmaceutically acceptable excipient. In some embodiments, the disease, the condition, or the disorder is selected from one or more of acne, sebaceous hyperplasia, and sebaceous adenitis. Any features described and/or claimed in relation to a method described herein apply mutatis mutandis to said compositions for use.

### BRIEF DESCRIPTION OF THE FIGURES

FIG. 1A is a graph showing the effect of initial cell confluency on total fatty acid production. Initial cell confluency (x-axis; C1, C2, C3, C4) is plotted against initial cell number (y-axis; t0 cell number (×10,000); lower plot). Initial cell confluency is also plotted against total fatty acid production of cells at each confluency as a percentage of total fatty acid production at confluency C1 (y-axis; Tot FA (% C1); upper plot).

FIG. 1B is a graph showing the correlation between total cell number at each initial cell confluency (x-axis) and total fatty acid production at each confluency as a percentage of total fatty acid production at confluency C1 (y-axis).

FIG. 1C is a bar graph showing the percent of fatty acids produced in response to insulin (C1+ins, C2+ins, C3+ins, and C4+ins) as compared to fatty acids produced in the absence of insulin at each cell confluency tested (C1-C4).

FIG. 2A is a Western blot (left panel) showing expression of epithelial membrane antigen (EMA), PPARγ, and β-actin (control) in SZ95 cells grown under serum-free (SZ95-SF) and 10% serum (SZ95-S) conditions for 24 hours. FIG. 2A also includes a bar graph (right panel) showing the EMA and PPARγ protein expression in SZ95-SF cells as a fraction of EMA and PPARγ protein expression in SZ95-S cells.

FIG. 2B is a bar graph showing total lipid (FA) production at 48 hours and 72 hours in SZ95-SF cells as a fraction of total lipid production in SZ95-S cells at the same time points.

FIG. 2C is a series of Western blots (left panel) showing protein expression of phosphorylated protein kinase B (pAkt), protein kinase B (Akt), phosphorylated S6 ribosomal protein (pS6), S6 ribosomal protein (S6), and β-actin (control) in SZ95-SF and SZ95-S cells cultured for 24 hours under control conditions (Ctr), or in the presence of 0.1 μM insulin (ins), the PPARγ modulator NAC-GED0507 (NAC-GED0507), or both insulin and NAC-GED0507 (NAC-GED0507 ins). FIG. 2C also includes a bar graph (right panel) showing fold change in expression of pS6 (light grey bars) and pAkt (dark gray bars) protein in SZ95-SF and SZ95-S cells treated with insulin (ins), NAC-GED0507, or both insulin and NAC-GED0507 (NAC-GED0507 ins), over corresponding untreated cells (Ctr).

FIGS. 3A-3E are a series of bar graphs showing mRNA expression levels (arbitrary units, normalized to GAPDH mRNA expression levels) of sterol response element-binding protein-1 (SREBP-1; FIG. 3A), fatty acid synthase (FAS; FIG. 3B), fatty acid delta-6-desaturase-2 (FADS-2; FIG. 3C), stearoyl-CoA desaturase-1 (SCD-1; FIG. 3D), and diglyceride acyltransferase (DGAT1; FIG. 3E) in SZ95-SF and SZ95-S cells cultured in the absence or presence of 0.1 μM insulin and/or 1 mM NAC-GED0507.

FIG. 4A is a bar graph showing fold change in the amount of saturated fatty acid (SFA), mono-unsaturated fatty acids (MUFA), and poly-unsaturated fatty acids (PUFA) in SZ95-SF cells compared to SZ95-S cells after 48 hours (light gray bars) or 72 hours (dark gray bars) in culture.

FIG. 4B is a bar graph showing fold change in the amount of total fatty acids in SZ95-SF cells (light gray bars) or SZ95-S cells (dark gray bars) treated with NAC-GED0507, insulin (ins), or both insulin and NAC-GED0507 (NAC-GED0507 ins) at 48 hours or 72 hours, compared to corresponding untreated cells (Ctr).

FIG. 4C is a pair of bar graphs showing fold change in percent of fatty acid composition for saturated fatty acids (SFA; white bars), mono-unsaturated fatty acids (MUFA; dark grey bars), and poly-unsaturated fatty acids (PUFA; light gray bars) in SZ95-SF cells compared to SZ95-S cells after 48 hours (left) or 72 hours (right) of treatment with insulin (ins) or insulin and NAC-GED0507 (NAC-GED0507 ins).

FIG. 5A is a bar graph showing LOX activity in SZ95-SF and SZ95-S cells exposed to insulin (ins; light gray bars) or insulin and NAC-GED0507 (ins+NAC-GED0507; dark gray bars) as a percent of LOX activity in corresponding untreated control cells.

FIG. 5B is a bar graph showing levels of 5-hydroxyeicosatetraenoic acid (5-HETE; left) and 15-hydroxyeicosatetraenoic acid (15-HETE; right) in SZ95-SF cells (light gray bars) and SZ95-S cells (dark gray bars) exposed to insulin (ins) or insulin and NAC-GED0507 (NAC-GED0507 ins) as a percent of 5-HETE and 15-HETE levels in corresponding untreated control cells.

FIG. 5C is a bar graph showing IL-6 released from SZ95-SF cells and SZ95-S cells exposed to insulin (ins; light gray bars) or insulin and NAC-GED0507 (ins+NAC-GED0507; dark gray bars) as a percent of IL-6 released from corresponding untreated control cells (IL-6 pg/cell number).

FIG. 6A is a Western blot (left panel) showing expression of EMA, PPARγ, and β-actin (control) in SZ95-SF cells cultured for 24 hours in the absence (Ctr) or presence of NAC-GED0507. FIG. 6A also includes a bar graph (right panel) showing fold change in EMA and PPARγ protein expression in SZ95-SF cells exposed to NAC-GED0507 over Ctr cells.

FIG. 6B is a bar graph showing expression of SREBP1, FADS2, and SCD1 mRNA in SZ95-SF cells treated with insulin (ins; light gray bars) or insulin and NAC-GED0507 (ins+NAC-GED0507; dark gray bars) as a percent of SREBP1, FADS2, and SCD1 mRNA, respectively, in untreated control (Ctr) cells.

FIG. 7 is a series of bar graphs showing (from left to right) mean Investigator's Static Global Assessment (ISGA) score, number of inflammatory lesions, and number of non-inflammatory lesions for patients enrolled in a phase I clinical trial evaluating treatment of acne with topical application of 1% NAC-GED0507 gel at day 0 of treatment (V1) or 3 weeks (V2) or 12 weeks (V5) after treatment.

FIG. 8A is a Western blot (left panel) showing levels of FADS2, PPARγ, and GAPDH (control) protein in extracts collected using Sebutape and D-Squame. FIG. 8A also includes a bar graph (right panel) showing fold change in levels of FADS2 and PPARγ protein levels in extracts collected using Sebutape (dark gray bars) or D-Squame (white bars) as compared to extracts collected using D-Squame.

FIG. 8B is a bar graph showing fold change in levels of squalene in extracts collected using Sebutape (dark gray bars) or D-Squame (white bars) compared to extracts collected using D-Squame.

FIG. 8C is a Western blot (left panel) showing levels of PPARγ and GAPDH (control) protein in sebum extracts collected from patients at day 0 of treatment (V1) or 3 weeks (V2) or 12 weeks (V5) after treatment with 1% NAC-GED0507 gel. FIG. 8C also includes a bar graph (right panel) showing PPARγ protein expression levels at V1, V2, and V5, as a ratio of PPARγ to GAPDH protein expression levels.

FIG. 8D is a Western blot (left panel) showing levels of pS6, S6, and GAPDH (control) protein in sebum extracts collected from patients at day 0 of treatment (V1) or 3 weeks (V2) or 12 weeks (V5) after treatment with 1% NAC-GED0507 gel. FIG. 8D also includes a bar graph (right panel) showing pS6 protein expression levels at V1, V2, and V5, as a ratio of pS6 to S6 normalized to GAPDH ((pS6/S6)/GAPDH) protein expression levels.

FIG. 9A is a bar graph showing levels of IL-1α protein in sebum collected using Sebutape (left) and in stratum corneum collected using D-Squame (right) at V1, V2, and V5 (shown as pg IL-1α protein/mg total protein).

FIG. 9B is a bar graph showing the level of lipid peroxidation in sebum collected at V1, V2, and V5, as determined by xylenol orange assay (shown as μg H₂O₂/mg sebum).

FIG. 9C is a bar graph showing amount of sapienic acid (C16:1 Δ6) as a percent of total sebum fatty acid methyl ester content (% Sebum C16:1) at V1, V2, and V5, as determined by GC-MS.

### DETAILED DESCRIPTION

The features and other details of the disclosure will now be more particularly described. Before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

“Preventing” includes delaying the onset of clinical symptoms, complications, or biochemical indicia of the state, disorder, disease, or condition developing in a subject that may be afflicted with or predisposed to the state, disorder, disease, or condition but does not yet experience or display clinical or subclinical symptoms of the state, disorder, disease, or condition. “Preventing” includes prophylactically treating a state, disorder, disease, or condition in or developing in a subject, including prophylactically treating clinical symptoms, complications, or biochemical indicia of the state, disorder, disease, or condition in or developing in a subject.

The term “pharmaceutically acceptable carrier” or “pharmaceutically acceptable excipient” as used herein interchangeably refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one biologically active compound as disclosed herein formulated together with one or more pharmaceutically acceptable excipients.

“Individual,” “patient,” or “subject” are used interchangeably and include to any animal, including mammals, preferably mice, rats, other rodents, rabbits, dogs, cats, swine, cattle, sheep, horses, or primates, and most preferably humans. The compounds of the invention can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). In some embodiments, the mammal treated in the methods of the invention is desirably a mammal in whom modulation of PPAR receptor activity, for example, a PPARγ receptor, is desired.

In the present specification, the term “therapeutically effective amount” means the amount of the subject compound that will elicit the biological or medical response of a tissue, system, animal or human that is being sought by the researcher, veterinarian, medical doctor, for example, a dermatologist, or other clinician. The compounds of the invention are administered in therapeutically effective amounts to treat and/or prevent a disease, condition, disorder, or state. Alternatively, a therapeutically effective amount of a compound is the quantity required to achieve a desired therapeutic and/or prophylactic effect, such as an amount which results in the prevention of or a decrease in the symptoms associated with a disease associated with modulation of PPAR receptor activation (e.g., PPARγ).

The term “pharmaceutically acceptable salt(s)” as used herein refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts. Pharmaceutically acceptable salts of the disclosure include, for example, pharmaceutically acceptable salts of N-acetyl-3-(4′-aminophenyl)-2-methoxypropionic acid, or a stereoisomer thereof, for example, pharmaceutically acceptable salts of N-acetyl-(S)-3-(4′-aminophenyl)-2-methoxypropionic acid.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

The compounds disclosed herein can exist in solvated as well as unsolvated forms with pharmaceutically acceptable solvents such as water, ethanol, and the like, and it is intended that the invention embrace both solvated and unsolvated forms.

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in the e.g., Examples herein by substituting an isotopically labeled reagent for a non-isotopically labeled reagent.

The disclosure provides methods for preventing acne in a patient, comprising administering topically to a patient a composition effective to induce sebocyte differentiation and/or to reduce insulin-induced LOX activity and/or inflammatory processes, where the composition comprises a therapeutically effective amount of a PPARγ modulator, and a pharmaceutically acceptable excipient. Also provided herein are methods of preventing acne and/or a disease, a condition, or a disorder characterized by alteration of sebocyte differentiation, comprising administering topically to a patient a composition effective to induce sebocyte differentiation, wherein the composition comprises a therapeutically effective amount of a PPARγ modulator, and a pharmaceutically acceptable excipient.

For example, in some embodiments, methods for preventing acne or preventing a disease, a condition, or a disorder characterized by alteration of sebocyte differentiation include methods of administering a pharmaceutically acceptable composition, for example, a pharmaceutically acceptable formulation, that includes one or more PPARγ modulators, to a patient. PPARγ modulator compounds can increase PPARγ activity and/or levels of PPARγ expression, for example, PPARγ mRNA and/or protein expression. Without wishing to be bound by theory, a PPARγ modulator can increase PPARγ activity by stimulating PPARγ binding to PPAR response elements (PPREs), stimulating PPARγ heterodimerization, stimulating PPARγ binding to retinoid X receptor, and/or stimulating PPARγ interaction with cofactors, for example, cofactors that increase the rate of transcription initiation.

Compounds useful in one or more of the disclosed methods are represented by formula I as depicted below (i.e., N-acetyl-3-(4′-aminophenyl)-2-methoxypropionic acid). Also provided herein are pharmaceutical compositions that include a compound represented by formula I; and a pharmaceutically or cosmetically acceptable excipient.

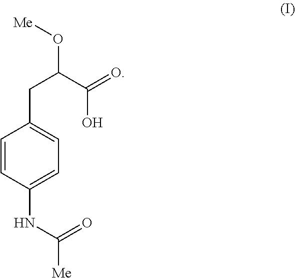 [SMILES: COC(Cc1ccc(NC(C)=O)cc1)C(=O)O]

In various embodiments, compounds and pharmaceutical compositions can include at least one compound selected from N-acetyl-(R)-3-(4′-aminophenyl)-2-methoxypropionic acid, and N-acetyl-(S)-3-(4′-aminophenyl)-2-methoxypropionic acid.

The compounds disclosed herein can be prepared in a number of ways well known to one skilled in the art of organic synthesis. Methods for making the compounds of formula I can be found, for example, in International Publication Nos. WO 2007/010516 and WO 2007/010514, each of which is incorporated by reference herein in its entirety.

The present disclosure also provides pharmaceutical compositions comprising compounds as disclosed herein formulated together with one or more pharmaceutically or cosmetically acceptable excipients. These formulations include those suitable for oral, rectal, topical, buccal and parenteral (e.g., subcutaneous, intramuscular, intradermal, or intravenous) administration, or for topical use, e.g., as part of a composition suitable for applying topically to skin and/or mucous membrane, for example, a composition in the form of a gel, a paste, a wax, a cream, a spray, a liquid, a foam, a lotion, an ointment, a topical solution, a transdermal patch, a powder, a vapor, or a tincture. Although the most suitable form of administration in any given case will depend on the degree and severity of the condition being treated and on the nature of the particular compound being used.

The present invention also provides a pharmaceutical composition comprising N-acetyl-3-(4′-aminophenyl)-2-methoxypropionic acid, or a pharmaceutically acceptable salt or a stereoisomer thereof (for example, N-acetyl-(S)-3-(4′-aminophenyl)-2-methoxypropionic acid); and optionally further comprising at least one compound selected from the group consisting of pioglitazone, rosiglitazone, doxycycline, hydroxychloroquine, mycophenolate mofetil, rifampin, clindamycin, and spermidine.

The present disclosure also provides methods that include the use of pharmaceutical compositions comprising compounds as disclosed herein (e.g., NAC-GED0507) formulated together with one or more pharmaceutically or cosmetically acceptable excipients. Exemplary compositions provided herein include compositions comprising essentially a PPARγ modulator, as described above, and one or more pharmaceutically acceptable excipients. Formulations include those suitable for oral, rectal, topical, buccal, and parenteral (e.g., subcutaneous, intramuscular, intradermal, or intravenous) administration, or for topical use, e.g., as a cosmetic product. The most suitable form of administration in any given case will depend on the clinical symptoms, complications, or biochemical indicia of the state, disorder, disease, or condition that one is trying to prevent in a subject; the state, disorder, disease, or condition one is trying to prevent in a subject; and/or on the nature of the particular compound and/or the composition being used.

### Therapeutic Applications

The disclosure is directed at least to methods for preventing acne or preventing a disease, a condition, or a disorder characterized by alteration of sebocyte differentiation by administering a pharmaceutically acceptable pharmaceutical composition, for example, a pharmaceutically acceptable pharmaceutical formulation, that includes one or more PPARγ modulators (e.g., NAC-GED0507), to a patient. For example, methods of preventing acne are provided, wherein a PPARγ modulator (or, e.g., a composition that includes a PPARγ modulator, for example, NAC-GED0507) is administered to a subject, for example, by topical administration. In some embodiments, methods of preventing a disease, condition, or disorder are provided, wherein a PPARγ modulator (or a composition that includes a PPARγ modulator, for example, NAC-GED0507) is administered to a subject, for example, by topical administration, and the disease, the condition, or the disorder is selected from non-inflammatory acne, inflammatory acne, acne vulgaris, acne fulminans, acne mechanica, acne conglobata, gram-negative folliculitis, pyoderma faciale, sebaceous hyperplasia, sebaceous adenitis, comedones (including whiteheads, blackheads, papules), pustules, nodules, cysts, cystic lesions, mild acne, moderate acne, severe nodulocystic acne. In some embodiments, the amount of the compound or composition administered is an amount that is effective to induce sebocyte differentiation and/or an amount that is effective to reduce insulin-induced LOX activity and/or inflammation.

Also provided herein are compositions for preventing acne or preventing a disease, a condition, or a disorder characterized by alteration of sebocyte differentiation. For example, in some embodiments, a disclosed composition includes a PPARγ modulator, for example NAC-GED0507, and one or more pharmaceutically acceptable excipients. Such compositions may be or may be part of, for example, a composition suitable for topical application, for example, a cream, a gel, or a foam such as an aerosol foam. Topical application includes application to the skin of a subject, for example, epidermis of a subject.

Compounds of the invention may be administered to subjects (e.g., animals and/or humans) in need of prophylactic treatment or prevention in dosages that will provide optimal pharmaceutical efficacy. It will be appreciated that the dose required for use in any particular application will vary from patient to patient, not only with respect to the particular compound or composition selected, but also with respect to the route of administration, the nature of the condition being treated, the age and condition of the patient, concurrent medication or special diets then being followed by the patient, and other factors which those skilled in the art will recognize, with the appropriate dosage ultimately being at the discretion of the attendant physician, caretaker, or patient. For preventing clinical conditions and diseases noted above, compounds of this invention may be administered, for example, orally, topically, parenterally, by inhalation spray, or rectally in dosage unit formulations containing conventional, non-toxic, pharmaceutically acceptable carriers, excipients, adjuvants, and vehicles. The term parenteral as used herein includes subcutaneous injections, intravenous, intramuscular, intrasternal injection, or infusion techniques.

Generally, a therapeutically effective amount of active component will be in the range of from about 0.1 mg/kg to about 100 mg/kg, from about 0.1 mg/kg to about 1 mg/kg, from about 0.1 mg/kg to about 10 mg/kg, from about 0.1 mg/kg to about 1000 mg/kg, from about 1 mg/kg to about 1000 mg/kg, from about 1 mg/kg to about 100 mg/kg, from about 1 mg/kg to 10 mg/kg, from about 10 mg/kg to about 20 mg/kg, from about 20 mg/kg to about 30 mg/kg, from about 30 mg/kg to about 40 mg/kg, from about 40 mg/kg to about 50 mg/kg, from about 50 mg/kg to about 60 mg/kg, from about 60 mg/kg to about 70 mg/kg, from about 70 mg/kg to about 80 mg/kg, from about 80 mg/kg to about 90 mg/kg, from about 90 mg/kg to about 100 mg/kg, from about 100 mg/kg to about 500 mg/kg, from about 500 mg/kg to about 1000 mg/kg, or from about 1000 mg/kg to about 2000 mg/kg. For example, a therapeutically effective amount of active component can be about 0.1 mg/kg, about 0.5 mg/kg, about 1 mg/kg, about 5 mg/kg about 10 mg/kg, about 20 mg/kg, about 30 mg/kg, about 40 mg/kg, about 50 mg/kg, about 60 mg/kg, about 70 mg/kg, about 80 mg/kg, about 90 mg/kg, about 100 mg/kg, about 200 mg/kg, about 300 mg/kg, about 400 mg/kg, about 500 mg/kg, or about 1000 mg/kg. The amount administered will depend on variables such as the type and extent of disease or indication to be treated, the overall health status of the particular patient, the relative biological efficacy of the compounds, formulations of compounds, the presence and types of excipients in the formulation, and the route of administration. The initial dosage administered may be increased beyond the upper level in order to rapidly achieve the desired blood-level or tissue level, or the initial dosage may be smaller than the optimum and the daily dosage may be progressively increased during the course of treatment depending on the particular situation. Human dosage can be optimized, e.g., in a conventional Phase I dose escalation study designed to run from 0.5 mg/kg to 20 mg/kg. Dosing frequency can vary, depending on factors such as route of administration, dosage amount, and the disease condition being treated. Exemplary dosing frequencies are once per day, twice per day, three times per day, once per week, twice per week, three times per week, four times per week, five times per week, six times per week, once every other day, once every three days, once every four days, once every five days, once every six days, once every eight days, once every nine days, once every ten days, once every eleven days, once every twelve days, once every thirteen days, and once every two weeks.

Formulations or compositions of the disclosure can comprise a disclosed compound and can also include a pharmaceutically acceptable excipient.

Compositions of the disclosure can be administered by various means, depending on their intended use, as is well known in the art. For example, if compositions of the present invention are to be administered orally, they can be formulated as tablets, capsules, granules, powders or syrups. Alternatively, formulations of the present invention can be administered parenterally as injections (intravenous, intramuscular, or subcutaneous), drop infusion preparations, enemas, or suppositories. For application by the ophthalmic mucous membrane route, compositions of the present invention can be formulated as eye drops or eye ointments. These formulations can be prepared by conventional means, and, if desired, the compositions can be mixed with any conventional excipient or additive, such as a binder, a disintegrating agent, a lubricant, a corrigent, a solubilizing agent, a suspension aid, an emulsifying agent or a coating agent.

Compositions of the disclosure can also be administered topically, for example, in the form of a cream, a gel, a solution, a foam, a spray, a paste, a wax, a liquid, a lotion, an ointment, a topical solution, a transdermal patch, a powder, a vapor, or a tincture. A topical formulation of the disclosure can be formulated to include one or more active components, for example NAC-GED0507, as a percent weight of the formulation. For example, a composition of the disclosure can include about 0.001%, about 0.01%, about 0.05%, about 0.1%, about 0.5%, about 1%, about 5%, about 10%, about 15%, about 20%, about 25%, or about 30% by weight of an active ingredient, for example, NAC-GED0507, where the percent weight indicates the percent weight of the active ingredient relative to the total weight of the formulation. A topical formulation of the disclosure can also be formulated to include one or more active components, for example NAC-GED0507, as a percent weight range of the formulation. For example, a composition of the disclosure can include about 0.001% to about 0.01% by weight, about 0.01% to about 0.1% by weight, about 0.1% to about 1% by weight, about 1% to about 5% by weight, about 5% to about 10% by weight, about 10% to about 20% by weight, or about 20% to about 30% by weight of an active ingredient, for example, NAC-GED0507, where the percent weight range indicates the percent weight range of the active ingredient relative to the total weight of the formulation.

In some embodiments of the formulations provided herein, wetting agents, emulsifiers, and lubricants, such as sodium lauryl sulfate and magnesium stearate, as well as coloring agents, release agents, coating agents, sweetening, flavoring, perfuming agents, preservatives, and antioxidants may be present in the formulated agents.

Subject compositions may be suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of composition that may be combined with a carrier material to produce a single dose may vary depending upon the subject being treated, and the particular mode of administration.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, film-coated tablets, sugar-coated tablets, powders, granules and the like), compositions of the disclosure may be mixed with one or more pharmaceutically acceptable excipients or carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration may include pharmaceutically acceptable emulsions, microemulsions, solutions, suspensions, syrups, and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

### Pharmaceutical Compositions and Routes of Administration

The present disclosure also provides methods for preventing acne or preventing a disease, a condition, or a disorder characterized by alteration of sebocyte differentiation by administering a pharmaceutical composition comprising one or more active compounds, e.g., a PPARγ modulator compound, e.g., NAC-GED0507, or a mixture thereof. In another aspect, the disclosure provides pharmaceutical compositions for use in preventing acne or preventing a disease, a condition, or a disorder characterized by alteration of sebocyte differentiation. Pharmaceutical compositions can be comprised of a disclosed PPARγ modulator compound, e.g., NAC-GED0507, and a pharmaceutically acceptable excipient. In embodiments, a pharmaceutical composition may be a mixture containing a specified amount of a therapeutic compound, e.g., a therapeutically effective amount, of a therapeutic compound, for example, a therapeutically effective amount of a PPARγ modulator compound (e.g., NAC-GED0507), in a pharmaceutically acceptable excipient for administering to a patient, e.g., a human, in order to treat, manage, ameliorate, and/or prevent acne or a disease, a condition, or a disorder characterized by alteration of sebocyte differentiation. In some embodiments, provided herein are pharmaceutical compositions comprising a disclosed PPARγ modulator compound (e.g., NAC-GED0507) and a pharmaceutically acceptable excipient. In some embodiments, the disclosure is directed to use of a PPARγ modulator compound (e.g., NAC-GED0507) in the manufacture of a medicament for treating, managing, ameliorating, and/or preventing acne or a disease, a condition, or a disorder characterized by alteration of sebocyte differentiation. “Medicament,” as used herein, has essentially the same meaning as the term “pharmaceutical composition.”

Pharmaceutically acceptable excipients may include buffers, carriers, and the like suitable for use in contact with the tissues of human beings and animals without excessive toxicity, irritation, allergic response, or other problem or complication, commensurate with a reasonable benefit/risk ratio. The excipient(s) should be “acceptable” in the sense of being compatible with the other ingredients of the formulations and not deleterious to the recipient. Pharmaceutically acceptable excipients include buffers, solvents, dispersion media, coatings, isotonic and absorption delaying agents, and the like, that are compatible with pharmaceutical administration. The use of such media and agents for pharmaceutically active substances is known in the art. In one embodiment the pharmaceutical composition is administered orally and includes an enteric coating or a lipophilic coating suitable for regulating the site of absorption of the encapsulated substances within the digestive system or gut. For example, an enteric coating can include an ethylacrylate-methacrylic acid copolymer, an amino alkyl methacrylate copolymer, a methacrylic acid copolymer, a methacrylic ester copolymer, an ammonioalkyl methacrylate copolymer, a polymethacrylate, a poly(methacrylic acid-co-methyl methacrylate), hydroxypropyl-methylcellulose phthalate.

In some embodiments, formulations provided herein include enteric coatings, for example, lipophilic coatings, that allow delivery of a therapeutic, for example, an isolated fatty acid, to one or more specific regions of the gastrointestinal tract. For example, formulations may include enteric coatings and reagents that allow delivery of therapeutic to the stomach, the duodenum, the jejunum, the small intestine, the large intestine, the transverse, ascending, or descending colon, the ileum, the cecum, and/or the rectum. Formulations may include enteric coatings and reagents that allow release of therapeutic from a formulation for oral administration in the form of, for example, a tablet, a lozenge, or a capsule, at an approximate pH value or within a pH value range. For example, formulations provided herein may include enteric coatings and reagents that release therapeutic, for example, an isolated fatty acid, from a formulation for oral administration at a pH value of about 3, about 4, about 4.5, about 5, about 5.5, about 6, about 6.5, about 7, about 7.5, or about 8. For example, formulations provided herein may include enteric coatings and reagents that release therapeutic from a formulation for oral administration at a pH value of greater than about 3, greater than about 4, greater than about 4.5, greater than about 5, greater than about 5.5, greater than about 6, greater than about 6.5, greater than about 7, greater than about 7.5, or greater than about 8. In some embodiments, formulations of the disclosure release therapeutic from a formulation for oral administration in a pH value range of about pH 3 to about pH, about pH 4 to about pH 5, about pH 5 to about pH 6, about pH 6 to about pH 7, about pH 7 to about pH 8, about pH 8 to about pH 9, about pH 4.5 to about pH 7.5, about pH 4 to about pH 7, about pH 5 to about pH 7, about pH 5.5 to about pH 6.5, or about pH 4.5 to about pH 5.5.

In some embodiments, a disclosed PPARγ modulator compound (e.g., NAC-GED0507) and any pharmaceutical composition thereof may be administered by one or several routes, including topically, parenterally, orally, pulmonarily, intratracheally, intranasally, transdermally, or intraduodenally. Parenteral administration includes subcutaneous injections, intrapancreatic administration, intravenous, intramuscular, intraperitoneal, intrasternal injection or infusion techniques. For example, a PPARγ modulator compound (e.g., NAC-GED0507) may be administered subcutaneously to a subject. In another example, a PPARγ modulator compound (e.g., NAC-GED0507) may be administered orally to a subject. In another example, a PPARγ modulator compound (e.g., NAC-GED0507) may be administered topically to the skin (e.g., the epidermis) or other body surface, for example to a specific region or regions of the skin (e.g., the back, the face, the cheeks, the forehead, the chin, the nose, the head, the neck, the shoulders, the arms, the legs, and/or the chest) via topical administration.

Pharmaceutical compositions containing a PPARγ modulator compound, such as those disclosed herein (e.g., NAC-GED0507), can be presented in a dosage unit form and can be prepared by any suitable method. A pharmaceutical composition should be formulated to be compatible with its intended route of administration. Useful formulations can be prepared by methods well known in the pharmaceutical art. For example, see Remington's Pharmaceutical Sciences, 18th ed. (Mack Publishing Company, 1990).

Pharmaceutical formulations, for example, are sterile. Sterilization can be accomplished, for example, by filtration through sterile filtration membranes. Where the composition is lyophilized, filter sterilization can be conducted prior to or following lyophilization and reconstitution.

### Topical Formulation and Use

The present disclosure provides topical formulations of a PPARγ modulator compound (e.g., NAC-GED0507) or a pharmaceutically acceptable salt or a stereoisomer thereof, including formulations that include a PPARγ modulator compound, or a pharmaceutically acceptable salt or a stereoisomer thereof, and a pharmaceutically acceptable excipient. In embodiments, the present disclosure provides a topical formulation of a PPARγ modulator compound (e.g., NAC-GED0507), or a pharmaceutically acceptable salt or a stereoisomer thereof, and one or more additional active ingredients, and a pharmaceutically acceptable excipient. For example, in embodiments, the present disclosure provides a topical formulation of a PPARγ modulator compound (e.g., NAC-GED0507), or a pharmaceutically acceptable salt or a stereoisomer thereof, one or more additional active ingredients for treating or preventing acne or a disease, a condition, or a disorder characterized by alteration of sebocyte differentiation; and a pharmaceutically acceptable excipient. In some embodiments, methods of treating or preventing acne or a disease, a condition, or a disorder characterized by alteration of sebocyte differentiation include administering a compound or formulation of the invention to a patient in combination with a therapy for treating or preventing acne or a disease, a condition, or a disorder characterized by alteration of sebocyte differentiation. Therapies for treating or preventing acne or a disease, a condition, or a disorder characterized by alteration of sebocyte differentiation include, but are not limited to, light-based therapies, laser therapies, photodynamic therapy, chemical peels (e.g., chemical peels that include one or more applications of a chemical solution, such as salicylic acid, glycolic acid, or retinoic acid), direct extraction of whiteheads and blackheads, and steroid injection.

In embodiments of the invention, active ingredients for treating or preventing acne or a disease, a condition, or a disorder characterized by alteration of sebocyte differentiation include, but are not limited to, retinoids and retinoid-like drugs, tretinoin, Retin-A, adapalene, tazarotene, antibiotics, antibiotics combined with benzoyl peroxide, clindamycin with benzoyl peroxide, erythromycin with benzoyl peroxide, benzoyl peroxide, a tetracycline antibiotic, minocycline, salicylic acid, azelaic acid, erythromycin, dapsone, zinc, sulfur, nicotinamide, resorcinol, sulfacetamide sodium, aluminum chloride, combined oral contraceptives that include estrogen and progestin, anti-androgen agents, antihistamines, spironolactone, isotretinoin, steroids, fish oil, brewer's yeast, probiotics, oral zinc, and topical tea tree oil.

In embodiments, the formulation of a PPARγ modulator compound (e.g., NAC-GED0507) may be formulated as a gel, a cream, an ointment, a liquid, or a patch dosage form. The formulation of a PPARγ modulator compound (e.g., NAC-GED0507) may be formulated such that upon applying to skin of a subject the formulation forms a patch.

For example, the topical formulations include or may include about 0.001% to about 0.01% by weight, about 0.01% to about 0.1% by weight, about 0.1% to about 1% by weight, about 1% to about 5% by weight, about 5% to about 10% by weight, about 10% to about 20% by weight, about 20% to about 30% by weight, or about 1% to about 10% by weight of a PPARγ modulator compound (e.g., NAC-GED0507), or a pharmaceutically acceptable salt or a stereoisomer thereof.

The topical formulations of the present disclosure may be formulated as a liquid, a solution, an emulsion, a cream, a lotion, a suspension, a triturate, a gel, a jelly, a foam, a paste, an ointment, a shampoo, an adhesive, a patch, a foam, an aerosol foam, or the like. In certain embodiments, the topical formulations of the present disclosure may be formulated as a liquid. In certain embodiments, the topical formulations of the present disclosure may be formulated as a solution. In certain embodiments, the topical formulations of the present disclosure may be formulated as an emulsion. In certain embodiments, the topical formulations of the present disclosure may be formulated as a cream. In certain embodiments, the topical formulations of the present disclosure may be formulated as a lotion. In certain embodiments, the topical formulations of the present disclosure may be formulated as a suspension. In certain embodiments, the topical formulations of the present disclosure may be formulated as a triturate. In certain embodiments, the topical formulations of the present disclosure may be formulated as a gel. In certain embodiments, the topical formulations of the present disclosure may be formulated as a jelly. In certain embodiments, the topical formulations of the present disclosure may be formulated as a foam. In certain embodiments, the topical formulations of the present disclosure may be formulated as a paste. In certain embodiments, the topical formulations of the present disclosure may be formulated as an ointment. In certain embodiments, the topical formulations of the present disclosure may be formulated as a shampoo. In certain embodiments, the topical formulations of the present disclosure may be formulated as an adhesive. In certain embodiments, the topical formulations of the present disclosure may be formulated as a patch. In certain embodiments, the topical formulations of the present disclosure upon application to a skin of a subject may form a patch.

The topical formulations (e.g., liquid, solution, emulsion, cream, lotion, suspension, triturate, gel, jelly, foam, past, ointment, shampoo, adhesive, patch, and the like) of a PPARγ modulator compound (e.g., NAC-GED0507), or a pharmaceutically acceptable salt or a stereoisomer thereof, are or may be suitable for treating, preventing, and/or ameliorating a acne or a disease, a condition, or a disorder characterized by alteration of sebocyte differentiation. In certain embodiments, the disease, the condition, or the disorder is selected from non-inflammatory acne, inflammatory acne, acne vulgaris, acne fulminans, acne mechanica, acne conglobata, gram-negative folliculitis, pyoderma faciale, sebaceous hyperplasia, sebaceous adenitis, comedones (including whiteheads, blackheads, papules), pustules, nodules, cysts, cystic lesions, mild acne, moderate acne, severe nodulocystic acne. In some embodiments, the amount of the compound or composition topically administered is an amount that is effective to induce sebocyte differentiation and/or an amount that is effective to reduce insulin-induced LOX activity when administered topically.

Daily topical administration of the PPARγ modulator compound (e.g., NAC-GED0507) or a pharmaceutically acceptable salt or a stereoisomer thereof, formulated as a topical formulation (e.g., a liquid, a solution, an emulsion, a cream, a lotion, a suspension, a triturate, a gel, a jelly, a foam, a paste, an ointment, a shampoo, an adhesive, a patch, and the like), once per day in the morning, and/or once per day in the evening during a treatment period which may be between one week, two weeks, one month, two months, or three months and one year may be effective to prevent and/or ameliorate acne or a disease, a condition, or a disorder characterized by alteration of sebocyte differentiation. The topical formulation (e.g., liquid, solution, emulsion, cream, lotion, suspension, triturate, gel, jelly, foam, past, ointment, shampoo, adhesive, patch, and the like) can be administered in an amount of, for example, between about 1.0 mL/5 cm² and 1.0 mL/50 cm², or between about 1.0 mL/5 cm² and 50 mL/50 cm², or between about 1.0 mL/5 cm² and 100 mL/50 cm².

In embodiments, the topical formulation (e.g., liquid, solution, emulsion, cream, lotion, suspension, triturate, gel, jelly, foam, past, ointment, shampoo, adhesive, patch, and the like) may be a mixture of both the PPARγ modulator compound (e.g., NAC-GED0507), or a pharmaceutically acceptable salt or a stereoisomer thereof, and a second therapeutic agent.

The topical formulation (e.g., liquid, solution, emulsion, cream, lotion, suspension, triturate, gel, jelly, foam, past, ointment, shampoo, adhesive, patch, and the like) of a PPARγ modulator compound (e.g., NAC-GED0507), or a pharmaceutically acceptable salt or a stereoisomer thereof, may also include one or more cosmetically or pharmaceutically acceptable carriers/excipients. Suitable carriers/excipients that may be used in the topical formulations discussed herein are known in the art and include, but are not limited to, solubilizers such as C₂ to C₈ straight and branched chain alcohols, diols and triols, moisturizers and humectants such as glycerine, amino acids and amino acid derivatives, polyamino acids and derivatives, pyrrolidone carboxylic acids and its salts and derivatives, surfactants such as sodium laureth sulfate, sorbitan monolaurate, emulsifiers such as cetyl alcohol, stearyl alcohol, thickeners such as methyl cellulose, ethyl cellulose, hydroxymethylcellulose, hydroxypropylcellulose, polyvinylpyrollidone, polyvinyl alcohol and acrylic polymers.

The topical formulation (e.g., liquid, solution, emulsion, cream, lotion, suspension, triturate, gel, jelly, foam, past, ointment, shampoo, adhesive, patch, and the like) of a PPARγ modulator compound (e.g., NAC-GED0507), or a pharmaceutically acceptable salt or a stereoisomer thereof, may also include propylene glycol. The propylene glycol may be present in the formulation between about 1% w/w to about 25% w/w. Additionally the topical formulation (e.g., liquids, solutions, emulsions, creams, lotions, suspensions, triturates, gels, jellies, foams, pastes, ointments, shampoos, adhesives, patches, and the like) of a PPARγ modulator compound (e.g., NAC-GED0507), or a pharmaceutically acceptable salt or a stereoisomer thereof, may also include ethanol and/or polyethylene glycol 300. The ethanol may be present in the formulation between about 1% w/w to about 25% w/w. The polyethylene glycol 300 may be present in the range of between about 1% w/w to about 80% w/w. In addition the topical formulation may include at least one moisturizer/humectant.

The topical formulation (e.g., liquid, solution, emulsion, cream, lotion, suspension, triturate, gel, jelly, foam, past, ointment, shampoo, adhesive, patch, and the like) of a PPARγ modulator compound (e.g., NAC-GED0507), or a pharmaceutically acceptable salt or a stereoisomer thereof, may be applied to the skin by any means known in the art including, but not limited to, by an aerosol, spray, pump-pack, brush, swab, or other applicator. In embodiments, the applicator provides either a fixed or variable metered dose application such as a metered dose aerosol, a stored-energy metered dose pump or a manual metered dose pump. In embodiments, the drug delivery system is applied to the skin of the human or animal covering a delivery surface area, for example, between about 10 and 800 cm², between about 10 and 400 cm², or between about 10 and 200 cm². The application may be performed by means of a topical metered dose spray combined with an actuator nozzle shroud which together accurately control the amount and/or uniformity of the dose applied. One function of the shroud may be to keep the nozzle at a pre-determined height above, and perpendicular to, the skin to which the drug delivery system is being applied. This function may also be achieved by means of a spacer-bar or the like. Another function of the shroud is to enclose the area above the skin in order to prevent or limit bounce-back and/or loss of the drug delivery system to the surrounding environment. In embodiments, the area of application defined by the shroud is substantially circular in shape.

In certain embodiments, the drug delivery system may be a unit volume dispenser with or without a roll-on or other type of applicator. It may also be necessary to apply a number of dosages on untreated skin to obtain the desired result.

Topical formulations (e.g., liquid, solution, emulsion, cream, lotion, suspension, triturate, gel, jelly, foam, past, ointment, shampoo, adhesive, patch, and the like) of a PPARγ modulator compound (e.g., NAC-GED0507), or a pharmaceutically acceptable salt or a stereoisomer thereof, of the present disclosure may contain a pharmaceutically acceptable topical excipient and a pharmacologically active base, without any additional pharmacologically active agents. The formulation may be may be prepared so as to contain liposomes, micelles, and/or microspheres. In certain embodiments, a topical formulation may be aqueous, i.e., contain water, or may be nonaqueous and optionally used in combination with an occlusive overlayer so that moisture evaporating from the body surface is maintained within the formulation upon application to the body surface and thereafter.

Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid. In embodiments, water-soluble ointment bases are prepared from polyethylene glycols of varying molecular weight; again, see Remington: The Science and Practice of Pharmacy for further information.

Creams, as also well known in the art, are viscous liquids or semisolid emulsions, either oil-in-water or water-in-oil. Cream bases are water-washable, and contain an oil phase, an emulsifier, and an aqueous phase. The oil phase, also called the “internal” phase, is generally included of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant.

Gels are semisolid, suspension-type systems. Single-phase gels contain organic macromolecules distributed substantially uniformly throughout the carrier liquid, which is typically aqueous, but also contains an alcohol and, optionally, an oil. In embodiments, “organic macromolecules,” i.e., gelling agents, are crosslinked acrylic acid polymers such as the “carbomer” family of polymers, e.g., carboxypolyalkylenes that may be obtained commercially under the CARBOPOL™ trademark. Hydrophilic polymers such as polyethylene oxides, polyoxyethylene-polyoxypropylene copolymers, and polyvinyl alcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin may also be included. In order to prepare a uniform gel, dispersing agents such as alcohol or glycerin can be added, or the gelling agent can be dispersed by trituration, mechanical mixing, or stirring, or combinations thereof.

Lotions are preparations to be applied to the skin surface without friction, and are typically liquid or semiliquid preparations in which solid particles, including the active agent, are present in a water or alcohol base. Lotions are usually suspensions of solids, and preferably, for the present purpose, include a liquid oily emulsion of the oil-in-water type. In embodiments, lotions are used for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like.

Pastes are semisolid dosage forms in which the PPARγ modulator compound (e.g., NAC-GED0507), or a pharmaceutically acceptable salt or a stereoisomer thereof, is suspended in a suitable base. Depending on the nature of the base, pastes are divided between fatty pastes or those made from a single-phase aqueous gels. The base in a fatty paste is generally petrolatum, hydrophilic petrolatum, or the like. The pastes made from single-phase aqueous gels generally incorporate carboxymethylcellulose or the like as a base.

Formulations may also be prepared with liposomes, micelles, and microspheres. Liposomes are microscopic vesicles having a lipid wall including a lipid bilayer, and can be used as drug delivery systems herein as well. Generally, liposome formulations are poorly soluble or insoluble pharmaceutical agents. Liposomal preparations for use in the instant disclosure may include cationic (positively charged), anionic (negatively charged), and neutral preparations. Cationic liposomes are readily available. For example, N[1-2,3-dioleyloxy)propyl]-N,N,N-triethylammonium (DOTMA) liposomes are available under the trade name LIPOFECTIN™. (ThermoFisher). Similarly, anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), and dioleoylphoshatidyl ethanolamine (DOPE), among others. These materials can also be mixed with DOTMA in appropriate ratios. Methods for making liposomes using these materials are well known in the art.

Micelles are known in the art as included of surfactant molecules arranged so that their polar head groups form an outer spherical shell, while their hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethylammonium chloride, dodecylammonium chloride, polyoxyl 8 dodecyl ether, polyoxyl 12 dodecyl ether, nonoxynol 10, and nonoxynol 30. Micelle formulations can be used in conjunction with the present disclosure either by incorporation into the reservoir of a topical or transdermal delivery system, or into a formulation to be applied to the body surface.

Microspheres, similarly, may be incorporated into the present formulations and drug delivery systems. Like liposomes and micelles, microspheres essentially encapsulate a drug or drug-containing formulation. Microspheres are generally, although not necessarily, formed from synthetic or naturally occurring biocompatible polymers, but may also be included of charged lipids such as phospholipids. Preparation of microspheres is well known in the art and described in the pertinent texts and literature.

Various additives, known to those skilled in the art, may be included in the topical formulations. For example, solvents, including relatively small amounts of alcohol, may be used to solubilize certain formulation components. In embodiment, the formulation includes a suitable enhancer, e.g., but are not limited to, ethers such as diethylene glycol monoethyl ether (available commercially as TRANSCUTOL™) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80), and lecithin (U.S. Pat. No. 4,783,450); alcohols such as ethanol, propanol, octanol, benzyl alcohol, and the like; polyethylene glycol and esters thereof such as polyethylene glycol monolaurate (PEGML); amides and other nitrogenous compounds such as urea, dimethylacetamide (DMA), dimethylformamide (DMF), 2-pyrrolidone, 1-methyl-2-pyrrolidone, ethanolamine, diethanolamine, and triethanolamine; terpenes; alkanones; and organic acids, particularly citric acid and succinic acid. AZONE™ and sulfoxides such as DMSO and C₁₀ MSO may also be used.

The present formulations may also include conventional additives such as opacifiers, antioxidants, fragrance, colorant, gelling agents, thickening agents, stabilizers, surfactants, and the like. Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof.

The formulations may also contain irritation-mitigating additives to minimize or eliminate the possibility of skin irritation or skin damage resulting from the pharmacologically active base or other components of the composition. Suitable irritation-mitigating additives include, for example: α-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. The irritant-mitigating additive, if present, may be incorporated into the present formulations at a concentration effective to mitigate irritation or skin damage, typically representing not more than about 20 wt. %, more typically not more than about 5 wt. %, of the composition.

The PPARγ modulator compound (e.g., NAC-GED0507), or a pharmaceutically acceptable salt or a stereoisomer thereof, may also be administered through the skin or mucosal tissue using a conventional skin patch, in which the PPARγ modulator compound (e.g., NAC-GED0507) is contained within a laminated structure that serves as a drug delivery device to be affixed to the body surface. In such a structure, the topical formulation is contained in a layer, or “reservoir,” underlying an upper backing layer. The laminated structure may contain a single reservoir, or it may contain multiple reservoirs.

In certain embodiments, the reservoir may include a polymeric matrix of a pharmaceutically acceptable adhesive material that serves to affix the system to the skin during delivery of a PPARγ modulator compound (e.g., NAC-GED0507), or a pharmaceutically acceptable salt or a stereoisomer thereof. In embodiments, the adhesive material may be a pressure-sensitive adhesive (PSA) that is suitable for long-term skin contact, and that is physically and chemically compatible with the PPARγ modulator compound (e.g., NAC-GED0507), or a pharmaceutically acceptable salt or a stereoisomer thereof, and any carriers, vehicles, or other additives that are present. Examples of suitable adhesive materials include, but are not limited to, the following: polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene). Preferred adhesives are polyisobutylenes.

The backing layer functions as the primary structural element of the transdermal system and provides the device with flexibility and, preferably, occlusivity. The material used for the backing layer should be inert and incapable of absorbing drug, base, or other components of the formulation contained within the device. The backing preferably includes a flexible elastomeric material that serves as a protective covering to prevent loss of drug and/or vehicle via transmission through the upper surface of the patch, and preferably imparts a degree of occlusivity to the system, such that the area of the body surface covered by the patch becomes hydrated during use. The material used for the backing layer should permit the device to follow the contours of the skin and be worn comfortably on areas of skin such as at joints or other points of flexure that are normally subjected to mechanical strain, with little or no likelihood of the device disengaging from the skin due to differences in the flexibility or resiliency of the skin and the device. The materials used as the backing layer are either occlusive or permeable, as noted above, although occlusive backings are preferred, and are generally derived from synthetic polymers (e.g., polyester, polyethylene, polypropylene, polyurethane, polyvinylidine chloride, and polyether amide), natural polymers (e.g., cellulosic materials), or macroporous woven and nonwoven materials.

The method of delivery of a topical formulation of a PPARγ modulator compound (e.g., NAC-GED0507), or a pharmaceutically acceptable salt or a stereoisomer thereof, may vary, but may involve application of a formulation of the disclosure to an area of body surface affected with or at risk of being affected with acne or a disease, a condition, or a disorder characterized by alteration of sebocyte differentiation. A cream, ointment, or lotion may be spread on the affected surface and gently rubbed in. A solution may be applied in the same way, but more typically will be applied with a dropper, swab, or the like, and carefully applied to the affected areas.

The dose regimen of a topical formulation of a PPARγ modulator compound (e.g., NAC-GED0507), or a pharmaceutically acceptable salt or a stereoisomer thereof, will depend on a number of factors that may readily be determined, such as severity or likely severity of the acne, or the disease, the condition, or the disorder characterized by alteration of sebocyte differentiation being treated, prevented, and/or ameliorated, and the responsiveness or likely responsiveness of the condition being treated, prevented, and/or ameliorated, but will normally be one or more doses per day, with a course of administration lasting from several days to several months. In general, the formulation can be applied one to four times daily. With a skin patch, the device is generally maintained in place on the body surface throughout a drug delivery period, typically in the range of 8 to 72 hours, and replaced as necessary.

Parenteral Administration

The pharmaceutical compositions of the disclosure can be formulated for parenteral administration, e.g., formulated for injection via the intravenous, intramuscular, subcutaneous, intralesional, or intraperitoneal routes. The preparation of an aqueous composition, such as an aqueous pharmaceutical composition containing a PPARγ modulator compound (e.g., NAC-GED0507), or a pharmaceutically acceptable salt or a stereoisomer thereof, will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. In addition, sterile, fixed oils may be employed as a solvent or suspending medium. For this purpose any bland fixed oil can be employed including synthetic mono- or diglycerides. In addition, fatty acids such as oleic acid can be used in the preparation of injectables. Sterile injectable preparations may also be sterile injectable solutions, suspensions, or emulsions in a nontoxic parenterally acceptable diluent or solvent, for example, as solutions in 1,3-butanediol. Among the acceptable vehicles and solvents that may be employed are water, Ringer's solution, U.S.P., and isotonic sodium chloride solution. In a particular embodiment, a fatty acid may be suspended in a carrier fluid comprising 1% (w/v) sodium carboxymethylcellulose and 0.1% (v/v) TWEEN™ 80. Under ordinary conditions of storage and use, these preparations contain a preservative to prevent the growth of microorganisms.

Injectable preparations, for example, sterile injectable aqueous or oleaginous suspensions may be formulated according to the known art using suitable dispersing or wetting agents and suspending agents. Generally, dispersions are prepared by incorporating the various sterilized active ingredients into a sterile vehicle which contains the basic dispersion medium and the required other ingredients from those enumerated above. Sterile injectable solutions of the disclosure may be prepared by incorporating a PPARγ modulator compound (e.g., NAC-GED0507), or a pharmaceutically acceptable salt or a stereoisomer thereof, in the required amount of the appropriate solvent with various of the other ingredients enumerated above, as required, followed by filtered sterilization. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The injectable formulations can be sterilized, for example, by filtration through a bacteria-retaining filter.

The preparation of more, or highly concentrated solutions for intramuscular injection is also contemplated. In this regard, the use of DMSO as solvent is preferred as this will result in extremely rapid penetration, delivering high concentrations of a PPARγ modulator compound (e.g., NAC-GED0507), or a pharmaceutically acceptable salt or a stereoisomer thereof, to a small area.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium 10 carbonate, sodium acetate, sodium biphosphate and the like, in amounts sufficient to maintain the pH at between about pH 6 and pH 8, and for example, between about pH 7 and pH 7.5. Suitable tonicity agents are dextran 40, dextran 70, dextrose, glycerin, potassium chloride, propylene glycol, sodium chloride, and the like, such that the sodium chloride equivalent of the solution is in the range 0.9 plus or minus 0.2%. Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone, carboxymethylcellulose and the like.

Oral Administration

In some embodiments, provided herein are compositions suitable for oral delivery of a PPARγ modulator compound (e.g., NAC-GED0507), or a pharmaceutically acceptable salt or a stereoisomer thereof, e.g., tablets that include an enteric coating, e.g., a gastro-resistant coating, such that the compositions may deliver a PPARγ modulator compound to, e.g., the gastrointestinal tract of a patient.

For example, a tablet for oral administration is provided that comprises granules (e.g., is at least partially formed from granules) that include a PPARγ modulator compound (e.g., NAC-GED0507), or a pharmaceutically acceptable salt or a stereoisomer thereof, and one or more pharmaceutically acceptable excipients. Such a tablet may be coated with an enteric coating. Tablets provided herein may include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, provided pharmaceutical formulations include an intra-granular phase that includes a PPARγ modulator compound (e.g., NAC-GED0507), or a pharmaceutically acceptable salt or a stereoisomer thereof, and a pharmaceutically acceptable filler. For example, a disclosed PPARγ modulator compound and a filler may be blended together, optionally, with other excipients, and formed into granules. In some embodiments, the intragranular phase may be formed using wet granulation, e.g. a liquid (e.g., water) is added to the blended PPARγ modulator compound and filler, and then the combination is dried, milled and/or sieved to produce granules. One of skill in the art would understand that other processes may be used to achieve an intragranular phase.

In some embodiments, provided formulations include an extra-granular phase, which may include one or more pharmaceutically acceptable excipients, and which may be blended with the intragranular phase to form a disclosed formulation.

A disclosed formulation may include an intragranular phase that includes a filler. Exemplary fillers include, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pre-gelatinized starch, calcium carbonate, and others including combinations thereof.

In some embodiments, a disclosed formulation may include an intragranular phase and/or a extragranular phase that includes a binder, which may generally function to hold the ingredients of the pharmaceutical formulation together. Exemplary binders of the disclosure may include, but are not limited to, the following: starches, sugars, cellulose or modified cellulose such as hydroxypropyl cellulose, lactose, pre-gelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others including combinations thereof.

Formulations of the disclosure, e.g., that include an intragranular phase and/or an extragranular phase, may include a disintegrant such as but are not limited to, starch, cellulose, crosslinked polyvinyl pyrrolidone, sodium starch glycolate, sodium carboxymethyl cellulose, alginates, corn starch, crosmellose sodium, crosslinked carboxymethyl cellulose, low substituted hydroxypropyl cellulose, acacia, and others including combinations thereof. For example, an intragranular phase and/or an extragranular phase may include a disintegrant.

In some embodiments, a provided formulation includes an intra-granular phase comprising a PPARγ modulator compound and excipients chosen from: mannitol, microcrystalline cellulose, hydroxypropylmethyl cellulose, and sodium starch glycolate or combinations thereof, and an extra-granular phase comprising one or more of: microcrystalline cellulose, sodium starch glycolate, and magnesium stearate or mixtures thereof.

In some embodiments, a provided formulation may include a lubricant, e.g., an extra-granular phase may contain a lubricant. Lubricants include but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In some embodiments, a pharmaceutical formulation comprises an enteric coating, for example, a lipophilic coating. Generally, enteric coatings create a barrier for the oral medication that controls the location at which the drug is absorbed along the digestive tract. Enteric coatings may include a polymer that disintegrates at different rates according to pH. Enteric coatings may include for example, cellulose acetate phthalate, methyl acrylate-methacrylic acid copolymers, cellulose acetate succinate, hydroxylpropylmethyl cellulose phthalate, methyl methacrylate-methacrylic acid copolymers, ethylacrylate-methacrylic acid copolymers, methacrylic acid copolymer type C, polyvinyl acetate-phthalate, and cellulose acetate phthalate.

Exemplary enteric coatings include Opadry® AMB, Acryl-EZE®, Eudragit® grades. In some embodiments, an enteric coating may comprise about 5% to about 10%, about 5% to about 20%, 8 to about 15%, about 8% to about 20%, about 10% to about 20%, or about 12 to about 20%, or about 18% of a tablet by weight. For example, enteric coatings may include an ethylacrylate-methacrylic acid copolymer.

For example, in some embodiments provided herein, a tablet is provided that comprises or consists essentially of about 0.5% to about 70%, e.g. about 0.5% to about 10%, or about 1% to about 20%, by weight of a PPARγ modulator compound (e.g., NAC-GED0507), or a pharmaceutically acceptable salt or a stereoisomer thereof. Such a tablet can include, for example, about 0.5% to about 60% by weight of mannitol, e.g., about 30% to about 50% by weight mannitol, e.g., about 40% by weight mannitol; and/or about 20% to about 40% by weight of microcrystalline cellulose, or about 10% to about 30% by weight of microcrystalline cellulose. For example, a disclosed tablet may comprise an intragranular phase that includes about 30% to about 60%, e.g. about 45% to about 65% by weight, or alternatively, about 5 to about 10% by weight of a PPARγ modulator compound, about 30% to about 50%, or alternatively, about 5% to about 15% by weight mannitol, about 5% to about 15% microcrystalline cellulose, about 0% to about 4%, or about 1% to about 7% hydroxypropylmethylcellulose, and about 0% to about 4%, e.g. about 2% to about 4% sodium starch glycolate by weight.

In another embodiment, a pharmaceutical tablet formulation for oral administration of a PPARγ modulator compound comprises an intra-granular phase, wherein the intra-granular phase includes a PPARγ modulator compound (e.g., NAC-GED0507), or a pharmaceutically acceptable salt or a stereoisomer thereof, and a pharmaceutically acceptable filler, and which may also include an extra-granular phase, that may include a pharmaceutically acceptable excipient such as a disintegrant. The extra-granular phase may include components chosen from microcrystalline cellulose, magnesium stearate, and mixtures thereof. The pharmaceutical composition may also include an enteric coating of about 12% to 20% by weight of the tablet. For example, a pharmaceutically acceptable tablet for oral use may include about 0.5% to 10% by weight of a PPARγ modulator compound, e.g., NAC-GED0507 or a pharmaceutically acceptable salt thereof, about 30% to 50% by weight mannitol, about 10% to 30% by weight microcrystalline cellulose, and an enteric coating comprising an ethylacrylate-methacrylic acid copolymer.

In another example, a pharmaceutically acceptable tablet for oral use may comprise an intra-granular phase, comprising about 5 to about 10% by weight of a PPARγ modulator compound (e.g., NAC-GED0507), or a pharmaceutically acceptable salt or a stereoisomer thereof, about 40% by weight mannitol, about 8% by weight microcrystalline cellulose, about 5% by weight hydroxypropylmethyl cellulose, and about 2% by weight sodium starch glycolate; an extra-granular phase comprising about 17% by weight microcrystalline cellulose, about 2% by weight sodium starch glycolate, about 0.4% by weight magnesium stearate; and an enteric coating over the tablet comprising an ethylacrylate-methacrylic acid copolymer.

In some embodiments the pharmaceutical composition may contain an enteric coating comprising about 13% or about 15%, 16%, 17% or 18% by weight, e.g., AcyrlEZE® (see, e.g., International Publication No. WO2010/054826, which is hereby incorporated by reference in its entirety).

The rate at which point the coating dissolves and the active ingredient is released is its dissolution rate. In an embodiment, a tablet may have a dissolution profile, e.g. when tested in a USP/EP Type 2 apparatus (paddle) at 100 rpm and 37° C. in a phosphate buffer with a pH of 7.2, of about 50% to about 100% of the PPARγ modulator compound releasing after about 120 minutes to about 240 minutes, for example after 180 minutes. In another embodiment, a tablet may have a dissolution profile, e.g. when tested in a USP/EP Type 2 apparatus (paddle) at 100 rpm and 37° C. in diluted HCl with a pH of 1.0, where substantially none of the PPARγ modulator compound is released after 120 minutes. A tablet provided herein, in another embodiment, may have a dissolution profile, e.g. when tested in USP/EP Type 2 apparatus (paddle) at 100 rpm and 37° C. in a phosphate buffer with a pH of 6.6, of about 10% to about 30%, or not more than about 50%, of the PPARγ modulator compound releasing after 30 minutes.

Formulations, e.g., tablets, in some embodiments, when orally administered to the patient may result in minimal plasma concentration of the PPARγ modulator compound in the patient. In another embodiment, disclosed formulations, when orally administered to a patient, topically deliver to the epidermis or skin of a patient, e.g., to an affected or diseased site of a patient, or a site that is likely to be diseased or affected by acne or a disease, a condition, or a disorder characterized by alteration of sebocyte differentiation (e.g., to prevent acne or a disease, a condition, or a disorder characterized by alteration of sebocyte differentiation).

In some embodiments, methods provided herein may further include administering at least one other agent that is directed to treatment of diseases and disorders disclosed herein. In some embodiments, other agents may be co-administered (e.g., sequentially or simultaneously).

Agents useful in the invention can include immunosuppressive agents including glucocorticoids, cytostatics, antibodies, agents acting on immunophilins, interferons, opioids, TNF binding proteins, mycophenolate, and small biological agents. For example, immunosuppressive agents include, but are not limited to: tacrolimus, cyclosporine, pimecrolimus, sirolimus, everolimus, mycophenolic acid, fingolimod, dexamethasone, fludarabine, cyclophosphamide, methotrexate, azathioprine, leflunomide, teriflunomide, anakinra, anti-thymocyte globulin, anti-lymphocyte globulin, muromonab-CD3, afutuzumab, rituximab, teplizumab, efalizumab, daclizumab, basiliximab, adalimumab, infliximab, certolizumab pegol, natalizumab, and etanercept. Other agents include antibiotics, anti-diarrheals, laxatives, pain relievers, other fatty acids, iron supplements, and calcium or vitamin D or B-12 supplements.

### Sebocyte Biology

The sebaceous gland and hair follicles together form the pilosebaceous unit of the skin. Sebocytes are the main cellular units of the sebaceous gland, which produces sebum—a mixture of cell debris, lipids, antimicrobial substances, free fatty acids, and matrix metalloproteinases. Sebum is a composed mainly of lipids, including: triglycerides, free fatty acids, wax esters, squalene, cholesterol esters, and cholesterol. Sebum functions to prevent excess water from entering the skin, while trapping and preventing necessary moisture from getting out. Additionally, sebum has antimicrobial and antioxidant delivery properties. While too little sebum can result in dry skin, excessive amounts of sebum results in clogging of the sebaceous gland and bacterial growth. The body reacts to bacterial growth caused by sebum blockage by mobilizing white blood cells. White blood cell mobilization in turn causes skin inflammation. Sebum synthesis is strongly regulated by hormone levels, in particular by androgens, which stimulate lipid production in sebocytes and sebocyte differentiation.

### Acne Pathology

The formulations and methods described herein are useful for preventing acne or a disease, a condition, or a disorder characterized by alteration of sebocyte differentiation in a patient. Acne and related conditions include, but are not limited to, non-inflammatory acne, inflammatory acne, acne vulgaris, acne fulminans, acne mechanica, acne conglobata, gram-negative folliculitis, pyoderma faciale, sebaceous hyperplasia, sebaceous adenitis, comedones (including whiteheads, blackheads, papules), pustules, nodules, cysts, cystic lesions, mild acne, moderate acne, and severe nodulocystic acne.

In general, acne occurs when hair follicles become clogged with dead skin cells and sebum, and/or as a result of excessive growth of the skin bacterium Propionibacterium acnes. Causes of acne include genetic predisposition, hormonal activity associated with puberty and female menstrual cycles (e.g., increased levels of androgens, testosterone, dihydrotestosterone (DHT), dehydroepiandrosterone (DHEA), growth hormone (GH), and insulin-like growth factor 1 (IGF-1)), bacterial overgrowth or infection, diet, stress, and environmental factors. Characteristics of acne include blackheads or whiteheads, pimples, oily skin, and scarring. Acne can present on areas of the skin, for example, the face, arms, shoulders, chest, head, and back.

Major pathogenic factors associated with acne are hyperkeratinization, obstruction of sebaceous follicles resulting from abnormal keratinization of the infundibular epithelium, stimulation of sebaceous gland secretion by androgens, and microbial colonization of pilosebaceous units by Propionibacterium acnes. In general, acne develops when a mixture of sebum and desquamated cells obstructs sebaceous follicles. The accumulation of sebum and cells distends the sebaceous follicle, forming a microcomedo, which expands to form an open or closed comedo. Without being bound by theory, P. acnes is believed to colonize and proliferate in the comedo, releasing inflammatory cytokines and chemotactic factors. This release of inflammatory cytokines and chemotactic factors weakens the follicular wall, causing it to rupture, and allowing the keratin-sebum mixture to leak into the dermis. This series of events results in the inflammatory lesions (e.g., papules, pustules, nodules, and cysts) associated with acne.

The body's inflammatory response is believed to play an important role in the pathogenesis of acne. For instance, early stage acne lesions are associated with lymphoid perivascular infiltrate, and accumulation of polymorphonuclear leukocytes continues in later stages of acne, causing distension, pustule formation, and, ultimately, lesion rupture. Additionally, P. acnes is believed to activate TLR2 in monocytes, resulting in proinflammatory cytokine release (including IL-12 and IL-8) and expression of antimicrobial peptides (including defensin-1, defensin-2, and defensin-3). Protease release by P. acnes also activates protease-activated receptor-2 (PAR-2) on keratinocytes, which enhances transcription of proinflammatory cytokines IL-1α, IL-8, and tumor necrosis factor-α (TNF-α), matrix metalloproteinases, and the cathelicidin LL-37.

Diet may play an important role in acne development as well. For instance, hyperglycemic carbohydrates, milk and dairy products, and saturated fats are increased in the Western diet and contribute to acne development. Insulin/insulin-like growth factor (IGF-1) signaling, along with branched-chain amino acids (BCAAs), glutamine, and palmitate, all of which are also generally increased in individuals who partake in a Western diet, and increase mammalian target of rapamycin complex 1 (mTORC1) signaling. Furthermore, increased mTORC1 activity in keratinocytes stimulates keratinocyte proliferation, pro-inflammatory NFκB signaling, increased transcription of pro-inflammatory cytokines (e.g., TNFα, IL-6, IL-8 IL-17, IL-20, IL-22 and IL-23), lipid biosynthesis, comedone development, and acne. Increased mTORC1 activity also promotes T cell activation and generation of CD4⁺ and CD8⁺ effector T cells. IGF-1 signaling also stimulates sterol response element binding protein-1 (SREBP-1) expression and lipogenesis in sebocytes via activation of the PI3K/AKT pathway, and IGF-1 suppresses nuclear Fox01 in sebocytes, which also contributes to increased lipogenesis.

### Acne Evaluation

Severity of acne and related disorders can be evaluated using different methods, including counting lesions and grading based on different observational criteria. Multiple grading systems exist, including the global acne grading system (GAGS), in which the face, chest, and back are divided into six areas and each area is assigned a factor based on its size. (See Doshi et al., (1997) Int J Dermatol 36:416-8.) Each type of lesion is given a value depending (e.g., no lesions=0, comedones=1, papules=2, pustules=3 and nodules=4), and the score for each area is calculated by multiplying the factor for each area by a severity grade of 0-4. The global score is the sum of each area's score. A score of 1-18 is considered mild, a score of 19-30 is considered moderate, a score of 31-38 is considered severe, and a score of greater than 39 is considered very severe. In the Investigator's Global Assessment (IGA) of acne severity grading system, a patient is assigned a score of 0-4 based on established criteria. In particular, a score of 0 (clear) indicates residual hyperpigmentation and potentially the presence of erythema; a score of 1 (almost clear) indicates a few scattered comedones and a few small papules; a score of 2 (mild) indicates that less than half the face is involved but that some comedones, papules, and pustules are observed; a score of 3 (moderate) indicates that more than half the face is involved, many comedones, papules, and pustules are present, and one nodule may be present; and a score of 4 (severe) indicates that the subject's entire face is involved, and is covered with comedones, numerous papules and pustules, and a few nodules and cysts. Methods of counting lesions generally require recording the number of each type of acne lesion and determining the overall severity of the lesions. Counting lesions or evaluating acne severity using, for example, the GAGS or IGA scoring systems, can be performed in connection with the methods described herein. A patient or patients without acne can be characterized as having a score of zero with either the GAGS or IGA scoring systems.

### Fatty Acids

Fatty acids are carboxylic acids with a long aliphatic chain, which is either saturated or unsaturated. In mammals, fatty acids usually exist as triglycerides, phospholipids, or cholesterol esters. Saturated fatty acids have no alkene bonds, while unsaturated fatty acids have one or more alkene bonds. Unsaturated fatty acids that include one alkene bond are known as monounsaturated fatty acids (MUFAs), while fatty acids that include more than one alkene bond are known as polyunsaturated fatty acids (PUFAs). Unsaturated fatty acids can be described by their chemical name, chemical structure, configuration (e.g., cis or trans) and number and position of double bonds, and/or the ratio of the total amount of carbon atoms of the fatty acid to the number of double (unsaturated) bonds. For example, myristoleic acid (chemical name) can also be described as CH₃(CH₂)₃CH═CH(CH₂)₇COOH (chemical structure); cis-Δ⁹ (cis configuration with a double bond positioned on the ninth carbon-carbon bond, counting from the carboxylic acid end of the compound); or 14:1 (a ratio of 14 total carbon atoms to 1 double bond). Other examples of unsaturated fatty acids include: palmitoleic acid (CH₃(CH₂)₅CH═CH(CH₂)₇COOH; cis-Δ⁹; 16:1), sapienic acid (CH₃(CH₂)₈CH═CH(CH₂)₄COOH; cis-Δ⁶; 16:1), oleic acid (CH₃(CH₂)₇CH═CH(CH₂)₇COOH; cis-Δ⁹; 18:1), elaidic acid (CH₃(CH₂)₇CH═CH(CH₂)₇COOH; trans-Δ⁹; 18:1), vaccenic acid (CH₃(CH₂)₅CH═CH(CH₂)₉COOH; trans-Δ¹¹; 18:1), linoleic acid (CH₃(CH₂)₄CH═CHCH₂CH═CH(CH₂)₇COOH; cis,cis-Δ⁹,Δ¹²; 18:2), linoelaidic acid (CH₃(CH₂)₄CH═CHCH₂CH═CH(CH₂)₇COOH; trans,trans-Δ⁹,Δ¹²; 18:2), α-linolenic acid (CH₃CH₂CH═CHCH₂CH═CHCH₂CH═CH(CH₂)₇COOH; cis,cis,cis-Δ⁹,Δ¹²,Δ¹⁵; 18:3), arachidonic acid (CH₃(CH₂)₄CH═CHCH₂CH═CHCH₂CH═CHCH₂CH═CH(CH₂)₃COOH; cis,cis,cis,cis-Δ⁵Δ⁸,Δ¹¹,Δ¹⁴; 20:4), eicosapentaenoic acid (CH₃CH₂CH═CHCH₂CH═CHCH₂CH═CHCH₂CH═CHCH₂CH═CH(CH₂)₃COOH; cis,cis,cis,cis,cis-Δ⁵Δ⁸,Δ¹¹,Δ¹⁴,Δ¹⁷; 20:5), erucic acid (CH₃(CH₂)₇CH═CH(CH₂)₁₁COOH; cis-Δ¹³; 22:1), and docosahexaenoic acid (CH₃CH₂CH═CHCH₂CH═CHCH₂CH═CHCH₂CH═CHCH₂CH═CHCH₂CH═CH(CH₂)₂COOH; cis,cis,cis,cis,cis,cis-Δ⁴,Δ⁷,Δ¹⁰,Δ¹³Δ¹⁶Δ¹⁹; 22:6). Free fatty acids in sebum, such as palmitic acid and oleic acid, stimulate IL-1β and IL-1α production and/or activity and comedogenesis.

Fatty acids such as arachidonic acid can be metabolized by 15-lipoxygenase-1 in human cells to form eicosanoids such as 15(S)-hydroperoxyeicosatetraenoic acid (15(S)-HpETE), which is further oxidized to form 15(S)-hydroxyeicosatetraenoic acid (15(S)-HETE). 15-lipoxygenase 2 metabolizes fatty acids, with a preference for linoleic acid, to form 15(S)-HpETE and 15(S)-HETE. 5-Hydroxyeicosatetraenoic acid (5(S)-HETE) is another eicosanoid metabolite of arachidonic acid. 15-lipoxygenase-1 metabolizes 5(S)-HETE to 5-(S),15-(S)-dihydroxy-eicosatetraenoate (5,15-diHETE). 12-Lipoxygenase metabolizes 5(S)-HETE to 5(S),12(S)-diHETE. 15(S)-HETE, 15(S)-HpETE, and 5(S)-HETE upregulate inflammatory responses.

### EXAMPLES

The embodiments described herein are further illustrated by the following examples. The examples are provided for illustrative purposes only, and are not to be construed as limiting the scope or content of the embodiments in any way.

### Example 1: Sebocyte Differentiation Under Serum-Free Conditions Results in Reduced Expression of Differentiation Markers

### Materials

Sebomed® basal medium was purchased from Merck-Biochrom (Berlin, Germany). Fetal bovine serum (FBS), L-glutamine, penicillin/streptomycin, recombinant human epidermal growth factor, trypsin/EDTA, and D-PBS were purchased from Invitrogen (Milan, Italy). Insulin was purchased from Sigma-Aldrich (Milan, Italy). SYBR Green PCR Master Mix was purchased from Bio-Rad (Milan, Italy). Antibodies to phosphoAkt (Ser473) (D9E, #4060), Akt (11E7, #4685), phospho-S6 (Ser235/236) (D57.2.2E, #4858), S6 (54D2, #2317), PPARγ (D9 #2430), anti-mouse IgG HRP-conjugated antibody, and anti-rabbit IgG HRP-conjugated antibody were purchased from Cell Signaling (Danvers, Mass., USA). Glyceraldehyde 3-phosphate dehydrogenase (GAPDH) antibody was purchased from Santa Cruz Biotechnology (Santa Cruz, Calif., USA). N-acetyl-(S)-3-(4′-aminophenyl)-2-methoxypropionic acid (“NAC-GED0507”) was supplied by Giuliani Pharma SpA, Milan Italy). IL-1alpha and IL-6 ELISA kit was purchased from Life Technologies (Italy).

### SZ95 Sebocyte Cell Culture

Immortalized human SZ95 sebocytes (Zouboulis et al., 1999), showing morphologic, phenotypic, and functional characteristics of normal human sebocytes, were cultured in 5% CO₂ at 37° C. in Sebomed basal medium, 1% L-glutamine, 1% penicillin/streptomycin, 5 ng/mL recombinant human epidermal growth factor, and 1 mM CaCl₂, with or without 10% FBS supplement. All experiments were performed at least in triplicate.

Lipid and Protein Extraction from SZ95 Sebocytes

Lipids were extracted from SZ95 sebocytes using a modified version of the protocol described in Bligh E G and Dyer W J, J Biochem Physiol, 1959. Lipids were extracted with chloroform: methanol (2:1) solution (2×1 mL) after the addition of butylhydroxytoluene to prevent oxidation of oxygen sensitive compounds. 5 μg of methylated tricosanoic acid was added as internal standard to control the analytical performance and to calculate the relative abundance of the fatty acids detected. Organic layers were collected and evaporated under nitrogen. Dried lipid extract was dissolved in 100 μL acetone/methanol/isopropanol (40:40:20) solution and 20 μL were used for the analysis of bound fatty acids (FA).

For protein extraction, cells were lysed in RIPA buffer (50 mM Tris-HCl pH 7.4, 1% Nonidet P40, 0.25% deoxycholate, 150 mM NaCl, 1 mM EDTA, 1 mM PMSF, 1 mM Na3VO4, 5 mM NaF), supplemented with a protease inhibitor cocktail (Roche, Mannheim, Germany), for 30 minutes at 4° C., then sonicated. Total cell lysates were clarified by centrifugation at 12,000 rpm for 10 minutes at 4° C. and then stored at −80° C. until analysis.

### Gas Chromatography-Mass Spectrometry (GC-MS) Analysis

Bound fatty acids (FA) were analyzed as FA methyl esters (FAME) obtained after derivatization. For simultaneous saponification and methylation of bound FA, 250 μL KOH solution (0.5 M) in anhydrous methanol was added to the dried extract and incubated at 37° C. for 20 minutes with constant shaking. 0.5 mL HCl (0.25 M) was added to neutralize the alkaline reaction mixture. After vortexing, 0.25 mL K₂SO₄ (6.7%) and 1 mL hexane:isopropanol (3:2 v/v) solution containing 0.0025% BHT were added and vortexed. After centrifugation, the lipid enriched upper phase was transferred to an Eppendorf tube and evaporated under nitrogen. The dried FAME extract was dissolved in 50 μL n-hexane and analyzed by GC-MS to establish FA profiles in the lipid extracts (Thermo-Finnigan, Waltham, Mass., USA). Chromatographic separation was carried out on a HP-FFAP capillary column (Agilent Technologies, Santa Clara, Calif., USA; crosslinked FFAP, length 50 m, film thickness 0.52 μm). Helium was used as the carrier gas. The initial GC oven temperature was 40° C. and was linearly ramped up to 240° C. at 8° C./min. The total run time was 60 min. The injector and the GC-MS transfer lines were kept at 230° C. and 250° C., respectively. Total ion chromatograms (TIC) were acquired, and areas of single peaks, corresponding to the FAME, were integrated with the qualitative analysis software. Identity of the detected FAME was verified by comparison with authentic standards and matched with library spectral data. GC-MS data is displayed as the mean±SD of three independent experiments performed in duplicate.

### Protein Quantification

Total amount of soluble protein was determined using the Pierce BCA Protein Assay Kit (ThermoScientific, Italy).

### Western Blot Analysis

Protein levels were measured by spectrophotometry, and protein (50 μg) was loaded and resolved using an acrylamide SDS-PAGE gel, and finally transferred onto a nitrocellulose membrane (Amersham Biosciences, Milan, Italy). Protein transfer efficiency was evaluated by Ponceau S staining (Sigma-Aldrich). After washing with PBS, the membranes were blocked with 5% fat-free dry milk in PBS with 0.05% Tween-20 for 1 hour at room temperature and then treated overnight at 4° C. with phospho-Akt, Akt, phospho-S6, S6, or PPARγ antibodies (dilution of 1:1000). A secondary anti-mouse IgG HRP-conjugated antibody (1:3000) and anti-rabbit IgG HRP-conjugated antibody (1:8000) were used for detection. Antibody complexes were visualized using ECL (Santa Cruz). Subsequent hybridization with GAPDH antibody (dilution of 1:5000) was used as a loading control. Protein levels were quantified by measuring optical density of specific bands using a UVI-TEC System (Eppendorf, Hamburg, Germany).

### Results

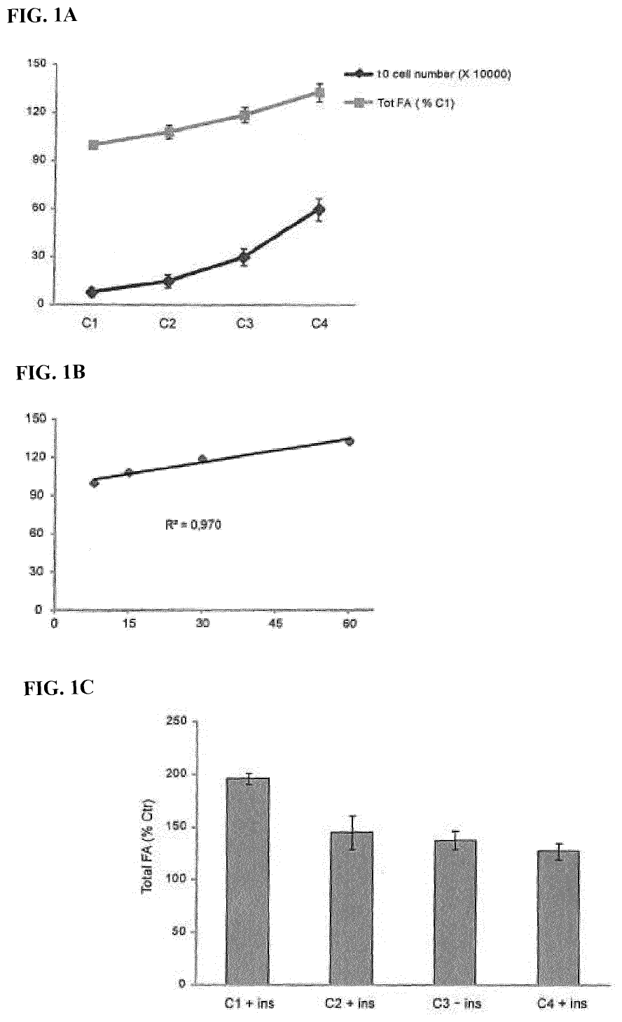

In order to analyze the effect of cell confluence on sebocyte differentiation and lipid production, SZ95 sebocytes were plated at different confluences (1×10⁵ cells, 1.5×10⁵ cells, 3×10⁵ cells, and 6×10⁵ cells) and the total amount of fatty acids was analyzed by GC-MS to determine lipid production. Total lipid production was used as an indicator of sebocyte differentiation. Sebocytes plated at higher confluences produced increasingly greater amounts of total fatty acids (FIG. 1A). Additionally, a direct correlation was observed between sebocyte confluence and total lipid production (FIG. 1). These results demonstrate that plating SZ95 sebocytes at a higher confluence resulted in greater total lipid production.

In order to analyze the effect of insulin stimulation on fatty acid production in sebocytes plated at different confluences, SZ95 sebocytes were plated at different confluences as described above and cultured in the presence or absence of 1p M insulin for 72 hours. Total fatty acid content was analyzed by GC-MS to determine lipid production. Insulin exposure produced a more dramatic increase in lipogenesis in sebocytes plated at the lowest tested confluence compared to sebocytes plated at higher confluence levels (FIG. 1C). These results indicate that insulin induces a greater increase in percent of fatty acid production over control levels in sebocytes cultured at lower confluences compared to sebocytes cultured at relatively higher confluences.

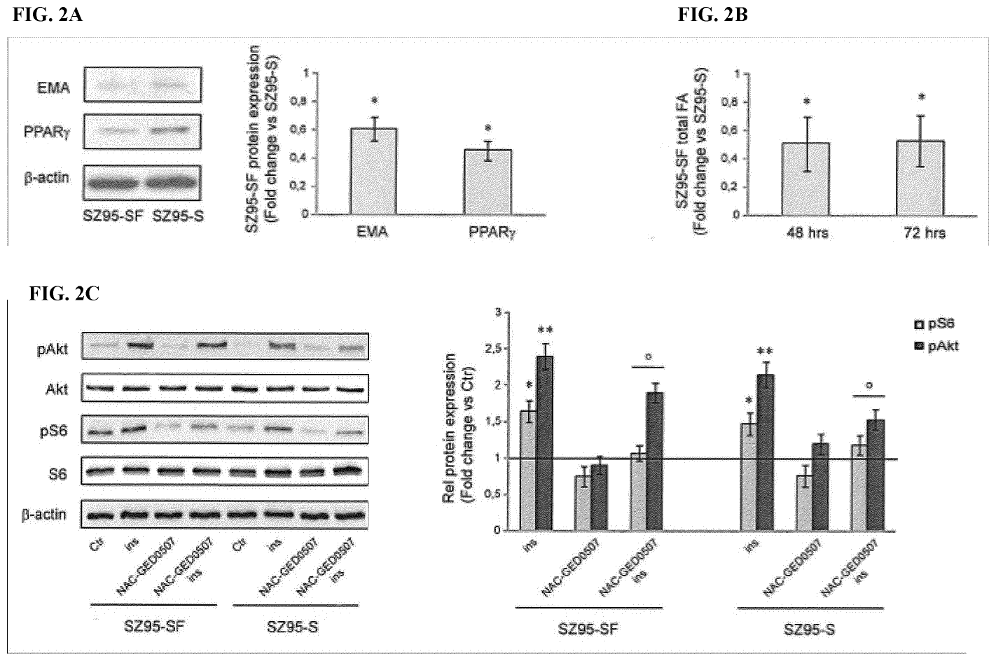

In order to determine whether serum conditions affect sebocyte differentiation, SZ95 human sebocytes were cultured in 10% serum (SZ95-S) or in serum-free (SZ95-SF) conditions for 24 hours, after which the expression of the human sebaceous differentiation marker epithelial membrane antigen (EMA) was analyzed by western blot. SZ95-SF cells showed a decrease in EMA expression levels relative to SZ95-S cells (FIG. 2A). SZ95-SF cells also showed decreased expression of PPARγ—which is implicated in sebocytes differentiation and lipid production—relative to SZ95-S cells (FIG. 2A). Evaluation of lipid production by GC-MS also demonstrated that SZ95-SF cells produced less lipid than SZ95-S cells after 48 hours and 72 hours of culture (FIG. 2B). These results demonstrate that when cultured in serum-free conditions, SZ95 sebocytes present underwent less differentiation as compared to SZ95 sebocytes cultured in the presence of serum.

### Example 2: N-acetyl-(S)-3-(4′-aminophenyl)-2-methoxypropionic acid Counteracts Insulin-Mediated Lipogenesis in Undifferentiated Human Sebocytes

Insulin and Insulin-like Growth Factor 1 (IGF-1) transmit lipogenic signals in sebocytes via the phosphoinositol-3-kinase (PI3K)/protein kinase B (Akt) pathway (Smith™, 2008). Akt and the S6 ribosomal protein, a downstream element of Akt pathway, are activated by phosphorylation. Levels of phosphorylated Akt (pAkt) and phosphorylated S6 ribosomal protein (pS6) are significantly increased by treatment with insulin (Mastrofrancesco et al., 2017).

To analyze the influence of insulin on Akt activation at different stages of sebocyte differentiation, SZ95 cells were cultured for 24 hours under SZ95-SF and SZ95-S conditions, as described above. The basal expression levels of pAkt and pS6 were significantly higher in SZ95-SF compared to SZ95-S sebocytes (FIG. 2C).

SZ95-SF and SZ95-S sebocytes were cultured for an additional 24 hours in the presence of 0.1 μM insulin and/or 1 mM of the PPARγ modulator N-acetyl-(S)-3-(4′-aminophenyl)-2-methoxypropionic acid (“NAC-GED0507”). Insulin exposure alone resulted in a greater increase in pAkt and pS6 levels in SZ95-SF sebocytes compared to SZ95-S sebocytes (FIG. 2C). Additionally, culturing sebocytes in the presence of 0.1 μM insulin and 1 mM of NAC-GED0507 resulted in a significant decrease in insulin-induced pAkt and pS6 in SZ95-SF cells (FIG. 2C). No significant changes in metabolic activity or cell viability were observed in SZ95-S or SZ95-SF sebocytes exposed to NAC-GED0507. These results demonstrate that basal levels of the activated insulin pathway signaling components pAkt and pS6 were increased in less differentiated SZ95-SF sebocytes relative to SZ95-S sebocytes; that insulin exposure resulted in a greater increase in activated insulin signaling components in less differentiated SZ95-SF sebocytes relative to SZ95-S sebocytes; and that the PPARγ modulator NAC-GED0507 countered the effects of insulin pathway activation in SZ95-SF sebocytes.

Overall, these results demonstrate that insulin induced a greater signaling response in less differentiated sebocytes as compared to more differentiated sebocytes cultured in the presence of serum. Additionally, NAC-GED0507 countered the effects of insulin pathway activation, and showed a more pronounced effect in less differentiated sebocytes as compared to more differentiated sebocytes cultured in the presence of serum.

### Example 3: NAC-GED0507 Counteracts Insulin-Induced Unsaturated Lipid Production in Undifferentiated Human Sebocytes

### RNA Extraction and Quantitative Real-Time PCR

Total RNA was isolated using the Aurum™ Total RNA Mini kit (Bio-Rad Laboratories Srl; Milan, Italy). Total RNA quantity, purity, and the absence of ribonuclease activity were assessed by OD260/280 absorbance measurements and by agarose gel electrophoresis. Total RNA samples were stored at −80° C. until use. Following DNAse I treatment, cDNA was synthesized using a mix of oligo-dT and random primers and RevertAid™ First Strand cDNA synthesis kit (Thermo Fisher Scientific; Monza, Italy). Quantitative real time RT-PCR was performed in a total volume of 15 μL with SYBR Green PCR Master Mix (Bio-Rad Laboratories Srl) and 200 nM concentration of each primer. Sequences of all primers used are indicated in Table 1.

TABLE 1
Primers used for Real time RT-PCR analysis

| mRNA | Oligonucleotide sequence (5′-3′) | SEQ ID NO |
|---|---|---|
| GAPDH | sense: TGCACCACCAACTGCTTAGC | SEQ ID NO: 1 |
| | antisense: GGCATGGACTGTGGTCATGAG | SEQ ID NO: 2 |
| SREBP-1 | sense: GGAGCCATGGATTGCACTTT | SEQ ID NO: 3 |
| | antisense: TCAAATAGGCCAGGGAAGTCA | SEQ ID NO: 4 |
| SCD-1 | sense: CATAATTCCCGACGTGGCTTT | SEQ ID NO: 5 |
| | antisense: AGGTTTGTAGTACCTCCTCTGGAACA | SEQ ID NO: 6 |
| FAS | sense: GACCGCTTCCGAGATTCC | SEQ ID NO: 7 |
| | antisense: GATGGCAGTCAGGCTCAC | SEQ ID NO: 8 |
| FADS-2 | sense: TGTCTACAGAAAACCCAAGTGG | SEQ ID NO: 9 |
| | antisense: TGTGGAAGATGTTAGGCTTGG | SEQ ID NO: 10 |
| PPARγ | sense: GCCAAGCTGCTCCAGAAAAT | SEQ ID NO: 11 |
| | antisense: TGATCACCTGCAGTAGCTGCA | SEQ ID NO: 12 |
| IL-6 | sense: AGCCACTCACCTCTTCAGAACG | SEQ ID NO: 13 |
| | antisense: GGTTCAGGTTGTTTTCTGCCAG | SEQ ID NO: 14 |
| DGAT-1 | sense: TCTACAAGCCCATGCTTCGAC | SEQ ID NO: 15 |
| | antisense: GGACGCTCACCAGGTACT | SEQ ID NO: 16 |

Reactions were carried out in triplicate using an iQ™ 5 iCycler supplied with the iQ™ 5 Real-Time Detection System (Bio-Rad Laboratories; Hercules, Calif., USA). Melt curve analysis was performed to confirm the specificity of the amplified products. Expression of mRNA species was normalized to the expression of GAPDH mRNA by the change in the Δ cycle threshold (ΔCt) method and calculated based on 2^{-Δct}.

### Results

To further analyze the effects of insulin stimulation on lipogenesis in human sebocytes, human sebocytes were cultured under SZ95-S or SZ95-SF conditions for 24 hours, as described above, and mRNA levels of genes regulating fatty acid de-saturation were then measured by real-time polymerase chain reaction (RT-PCR). Basal mRNA expression of fatty acid delta-6-desaturase-2 (FADS-2) and stearoyl-CoA desaturase-1 (SCD-1) were significantly higher in SZ95-SF sebocytes compared to SZ95-S sebocytes.

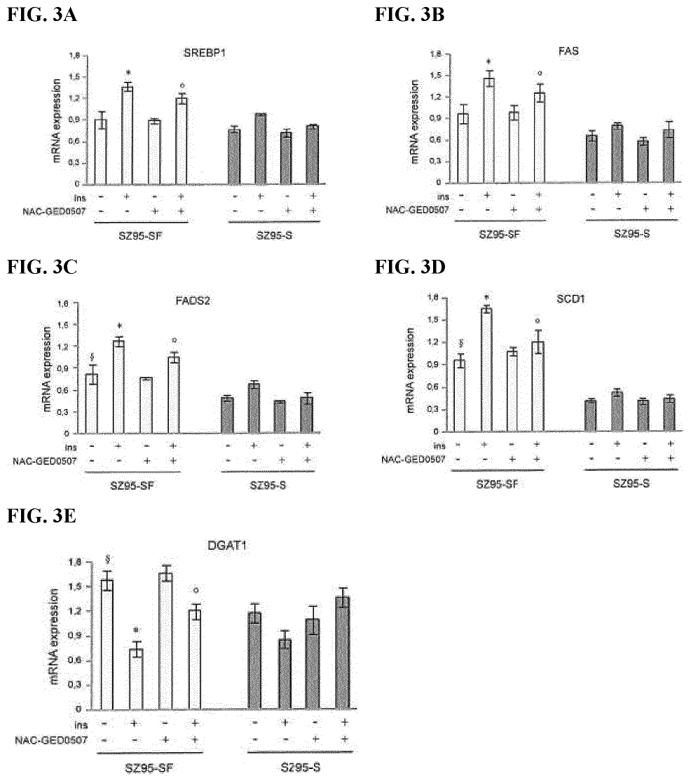

SZ95-S and SZ95-SF sebocytes were cultured for an additional 24 hours in the presence of 0.1 μM insulin and/or 1 mM NAC-GED0507. Exposure to insulin alone resulted in a significant increase in the expression levels of genes involved in de novo synthesis and de-saturation of fatty acids, including the sterol response element-binding protein-1 (SREBP-1), fatty acid synthase (FAS), FADS-2, and SCD-1 (FIGS. 3A-3D). Significant increases in expression of this group of genes were observed in SZ95-SF sebocytes exposed to insulin, but not in SZ95-S sebocytes exposed to insulin. Notably, exposing cells to both insulin and NAC-GED0507 significantly inhibited the up-regulation of lipidogenic genes observed in the presence of insulin alone (FIGS. 3A-3D).

By contrast, treating SZ95-SF cells with 0.1 μM insulin resulted in a significant decrease in mRNA levels of diglyceride acyltransferase (DGAT1), while treatment with the PPARγ modulator NAC-GED0507 countered the effects of insulin exposure (FIG. 3E).

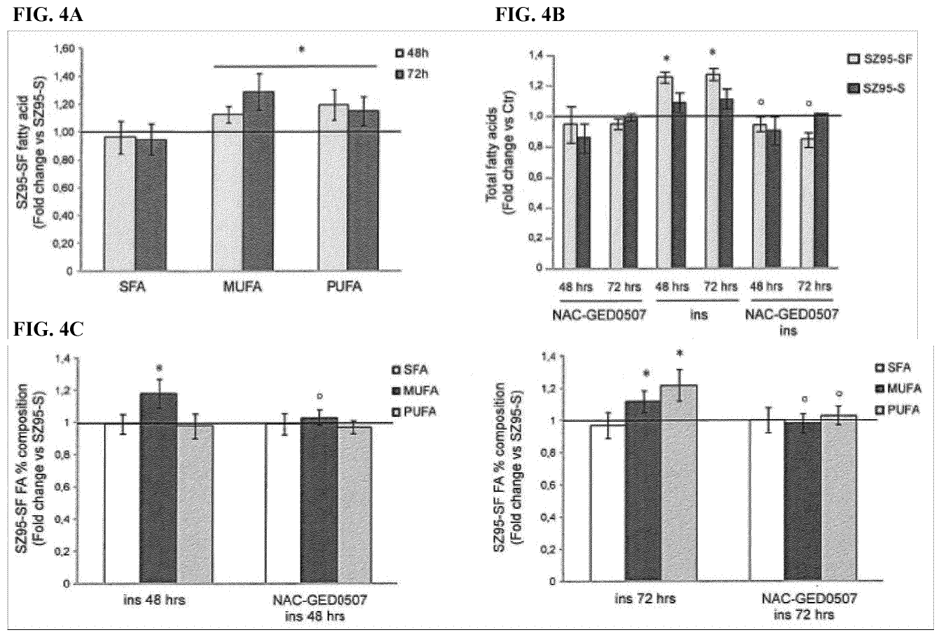

Sebocytes were also cultured under SZ95-SF and SZ95-S conditions for 48 hours or 72 hours, and GC-MS analysis was performed to detect the level of total lipids. SZ95-SF sebocytes cultured for 48 hours or 72 hours expressed lower levels of total lipid content compared to SZ95-S sebocytes cultured for the same amount of time, as assessed by fatty acid methyl ester (FAME) content of SZ95 lipid extracts (FIG. 2B). Further analysis revealed that the content of mono-unsaturated fatty acids (MUFA) and poly-unsaturated fatty acids (PUFA) was greater in SZ95-SF sebocytes as compared to SZ95-S sebocytes at both 48 hours and 72 hours (FIG. 4A; SFA=saturated fatty acid).

Exposure of sebocytes to insulin resulted in a significant and larger increase in total lipogenesis in SZ95-SF sebocytes at 48 hours and 72 hours, as compared SZ95-S sebocytes (FIG. 4B). Insulin stimulation also produced a greater increase in production of mono-unsaturated fatty acids after 48 h and in mono- and poly-unsaturated fatty acids after 72 h in SZ95-SF sebocytes, as compared to SZ95-S sebocytes (FIG. 4C). These results demonstrate that insulin stimulated lipogenesis in sebocytes, with a significant effect on production of fatty acid production in less differentiated SZ95-SF cells. These results also demonstrate that insulin stimulated more robust production of unsaturated fatty acids in less differentiated SZ95-SF sebocytes as compared to SZ95-S sebocytes.

SZ95-SF and SZ95-S sebocytes were also cultured for 48 hours or 72 hours in the presence of insulin and NAC-GED0507. The presence of NAC-GED0507 significantly inhibited the total fatty acid synthesis stimulated by insulin exposure in SZ95-SF sebocytes (FIG. 4B). The presence of NAC-GED0507 also significantly inhibited the relatively more robust production of mono-unsaturated fatty acids and poly-unsaturated fatty acids observed in SZ95-SF sebocytes in response to insulin, and inhibition observed in SZ95-SF sebocytes was relatively more robust in comparison to inhibition observed in SZ95-S sebocytes (FIG. 4C). These results demonstrate that PPARγ modulation by NAC-GED0507 significantly counteracted the insulin-induced increase in total lipogenesis and unsaturated bound fatty acid production observed in less differentiated SZ95-SF sebocytes.

### Example 4: NAC-GED0507 Counteracts Insulin-Induced Lipoxygenase Activity in Undifferentiated Human Sebocytes

### Lipoxygenase Activity Assay

Lipoxygenase (LOX) activity was evaluated spectrophotometrically by monitoring the conversion of the LOX substrate linoleic acid (LA) to the corresponding hydroperoxy fatty acid. SZ95 sebocytes were lysed in 50 mM Tris/HCl buffer (pH 7.4) (Merck; Darmstadt, Germany) by repeated freezing in liquid nitrogen and thawing. Protein content was determined by Bradford assay using bovine serum albumin as the protein standard. Aliquots of supernatants containing 50 μg of protein were added to Tris/HCl buffer containing 200 μM LA in a final volume of 1 mL. All reactions were performed in 1-cm path length quartz cuvettes. The absorbance at 234 nm was recorded at room temperature for 10 minutes using a Lambda 25 UV/Vis spectrophotometer (Perkin-Elmer, UK).

### Liquid Chromatography-Mass Spectrometry (LC-MS) Analysis

Levels of the polyunsaturated fatty acids 5-hydroxyeicosatetraenoic acid (5-HETE) and 15-hydroxyeicosatetraenoic acid (15-HETE) in cell media were measured by HPLC\MS\MS. Chromatographic separation was carried out using an Agilent Technologies 1200 HPLC Liquid Chromatography System (Palo Alto, Calif., USA) with a Symmetry C18 column (3.5 m, 100 mm×2.1 mm; Waters, Milford, Mass., USA) as previously reported (Furugen A et al., (2015) “Simultaneous quantification of leukotrienes and hydroxyeicosatrienoic acids in cell culture medium using liquid chromatography/tandem mass spectrometry” Biomed Chromatogr 29:1084-1093). Negative ion electrospray tandem mass spectrometry was carried out with an Agilent Technologies triple quadrupole 6400 Series mass spectrometer at unit resolution with multiple reaction monitoring (MRM) performed by monitoring the following transitions: 5-HETE 319→115; 5-HETE-d8 (IS for 5-HETE) 327→116; 15-HETE 319→219; and 15-HETE-d8 (IS for 15-HETE). For each compound to be quantified, an internal standard was selected and a linear curve was generated where the ratio of analyte standard peak area to internal standard peak area was plotted against the amount of analyte standard. The results were calculated as nM/106 cells and then reported as % variation vs control values.

### Results

LOX activity metabolizes LA—a preferred FADS-2 substrate—allowing FADS-2 to desaturate palmitate, which in turn results in increased sapienate production. (Ge L et al, 2003; Sapienic Acid: Species-Specific Fatty Acid Metabolism of the Human Sebaceous Gland, Chapter 10, Lipids and skin health, Pappas A Editor, Springer, 2014.) LOX oxidation of LA and its metabolite—arachidonic acid—produces oxidized poly-unsaturated fatty acids that are likely involved in the inflammation pathway observed in acne lesions. For example, increased levels of 5-lipoxygenase (5-LOX) have been detected in skin of acne patients, and treatment with a 5-LOX inhibitor reduces inflammatory lesions and suppresses production of pro-inflammatory lipids such as hydroperoxides (Alestas et al., J Mol Med, 2003; Zouboulis C C et al., Arch Dermatol, 2003; Zouboulis C C et al., Dermatology, 2003; Zouboulis C C et al., Exp Dermatol, 2010).

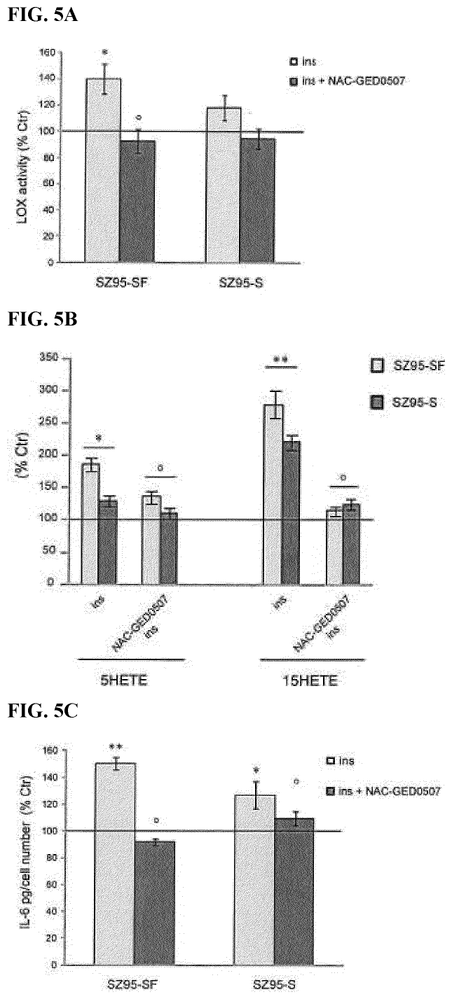

SZ95 sebocytes cultured under serum and serum-free conditions were analyzed for LOX expression. SZ95-SF sebocytes expressed slightly higher basal levels of LOX compared to SZ95-S sebocytes (data not shown). Exposure of SZ95-SF and SZ95-S to 0.1 μM insulin resulted in a significant and larger increase in LOX activity in SZ95-SF cells compared to SZ95-S cells, as measured by LA metabolism. Additionally, exposing sebocytes to both insulin and NAC-GED0507 inhibited the insulin-induced increase in LOX activity observed in SZ95-S and SZ95-SF cells (FIG. 5A).

Levels of the polyunsaturated fatty acids 5-HETE and 15-HETE were also measured in SZ95-SF and SZ95-S sebocytes by LC-MS. Following insulin treatment, higher levels of 5-HETE and 15-HETE and a larger percent increase in 5-HETE and 15-HETE production were measured in SZ95-SF cells compared to SZ95-S cells (FIG. 5B). Additionally, exposure to NAC-GED0507 countered the increase in 5-HETE and 15-HETE stimulated by insulin exposure (FIG. 5B). This data demonstrates that, in addition to stimulating lipogenesis and fatty acid desaturation, insulin stimulated a greater increase in LOX activity in less differentiated sebocytes compared to differentiated sebocytes. These results also demonstrate that PPARγ modulation by NAC-GED0507 counteracted the effects of insulin stimulation on LOX activity and polyunsaturated fatty acid production in sebocytes.

### Example 5: NAC-GED0507 Inhibits Insulin-Induced IL-6 Release in Undifferentiated Sebocytes

### Cytokine Analysis

SZ95 culture supernatants were collected and centrifuged to remove cell debris. Aliquots were then stored at −80° C. until use. IL-6 protein level was determined by ELISA (Life Technologies, Invitrogen; Milan, Italy), and normalized for cell number. In stratum corneum specimens, concentration of IL-1α was assessed by ELISA (Life Technologies, Invitrogen, Milan, Italy), and normalized against the total amount of soluble protein. Levels of IL-1α were reported as pg/pg protein. The results represent the average of three independent experiments performed in triplicate.

### Results

To determine the effect of insulin exposure on inflammatory cytokine release in sebocytes at different stages of differentiation, IL-6 release was measured by ELISA following exposure of SZ95-SF and SZ95-S sebocytes to 0.1 μM insulin. Insulin treatment resulted in greater IL-6 release in SZ95-SF sebocytes compared to SZ95-S sebocytes (FIG. 5C). Furthermore, addition of NAC-GED0507 significantly reduced the amount of IL-6 cytokine release in all sebocytes (FIG. 5C). These results demonstrate that insulin stimulation produced greater levels of inflammatory cytokine release in relatively less differentiated sebocytes compared to more differentiated sebocytes, and that PPARγ modulation by NAC-GED0507 counteracted insulin-stimulated inflammatory cytokine release in sebocytes.

Overall, the foregoing results demonstrate that insulin exposure in less differentiated SZ95 sebocytes mimicked features of acne pathology, including induction of sebogenesis with a large mono-unsaturated fatty acid component, induction of lipoxygenase activity, and induction of inflammatory cytokine release. Additionally, insulin exposure induced a less pronounced response in more highly differentiated SZ95 cells cultured in the presence of serum. Furthermore, the PPARγ modulator NAC-GED0507 significantly inhibited the effects of insulin exposure in sebocytes.

### Example 6: Pre-Treatment with NAC-GED0507 Counteracts Insulin Stimulation in SZ95-SF Cells by Induction of Cell Differentiation

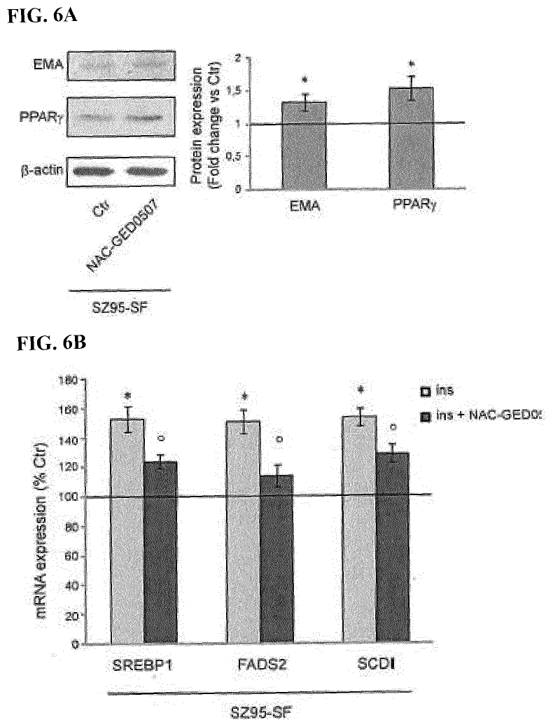

To evaluate whether NAC-GED0507 promotes sebocyte differentiation and, in turn renders cells less sensitive to insulin challenge, SZ95-SF cells were cultured for 24 hours with or without NAC-GED0507 treatment. Western blot analysis demonstrated that NAC-GED0507 induced an increase in protein expression of both PPARγ and EMA (FIG. 6A). This result indicates that a PPARγ modulator induces PPARγ expression as well as expression of protein (e.g., EMA) indicative of advanced sebocyte differentiation. Pre-treatment of SZ95-SF cells with NAC-GED0507 also reduced the effect of insulin-stimulated SREBP1, FADS2, and SCD1 mRNA expression in SZ95-SF cells (FIG. 6B). These results indicate that the PPARγ modulator NAC-GED0507 promoted sebocyte cell differentiation, thereby inhibiting insulin-induced lipogenesis.

### Example 7: Topical Treatment with NAC-GED0507 is Effective in the Treatment of Mild to Moderate Acne

### Study Participants

Twenty-one patients with mild-to moderate facial acne were recruited from the Acne Ambulatory for an in vivo open label Phase I clinical trial assessing the topical treatment of acne with 1% NAC-GED0507 (EUDRACT NUMBER: 2014-005244-17). The study was approved by the Institutional Ethical Committee and adhered to the Declaration of Helsinki principle guidelines. Study subjects provided informed consent prior to their participation. Enrolled patients underwent a dermatological examination at day 0 (V1), 3 weeks (V2), 6 weeks (V3), 9 weeks (V4), and 12 weeks (V5) after treatment began. Acne lesion count (overall, inflammatory, and non-inflammatory) and Investigator's Static Global Assessment (ISGA) score were determined at each time point up to V5. At V1, V2, and V5, sebum and stratum corneum withdrawal was also performed. Patients were instructed to apply an amount of product sufficient to cover the entire facial surface once a day, either in the morning or at night, depending on their personal habits. The gel formulation was supplied by Giuliani S.p.A. (Milan, Italy).

### Sampling

Sebum samples were collected from the foreheads of patients using Sebutapes™ (Cuderm, Dallas, Tex., USA) weighed beforehand as previously described (Camera E, et al., (2010) “Comprehensive analysis of the major lipid classes in sebum by rapid resolution high-performance liquid chromatography and electrospray mass spectrometry.” J Lipid Res 51(11):3377-88; Capitanio B, et al., (2014) “Modulation of sebum oxidation and interleukin-la levels associates with clinical improvement of mild comedonal acne” JEADV 28(12):1792-7). Skin was cleaned gently with 70% ethanol and three pieces of tape were applied onto patient foreheads for 30 minutes. The tapes were weighed after sampling to assess the amount of sebum collected. Sebum excretion rate was calculated and expressed as μg/cm²/min. Stratum corneum was sampled from lesional and non-lesional areas by tape stripping with D-Squame (Cuderm, Dallas, Tex., USA; as previously described in de Jongh C M, et al., (2007) “Cytokines at different stratum corneum levels in normal and sodium lauryl sulphate-irritated skin” Skin Res Technol, 13(4):390-8; Capitanio B, et al., (2014) JEADV).

Sebum lipids were extracted from Sebutapes™ with ethanol (Merck, Darmstadt, Germany) containing 0.025% of butylhydroxytoluene (BHT; Sigma-Aldrich, Milan, Italy) to prevent oxidation, and further cleaned by liquid-liquid extraction with ethyl acetate. The extract was dissolved in an acetone/methanol/isopropanol (40/40/20, v/v) mixture at a concentration of 5 mg/mL and stored at −80° C. until analysis. Proteins extraction was performed by scraping each tape in 150 μl of buffer containing Tris-HCl, pH=6.8; SDS 10%; and protease inhibitor. Extracts were centrifuged (10 minutes at 10,000 g), and supernatant aliquots were frozen at −80° C. until analysis.

Soluble proteins were extracted according to de Jongh C M, et al., (2007) Skin Res Technol, with slight modifications. 250 μL of phosphate-buffered saline (Euroclone, Milan, Italy) with 0.005% Tween-20 (Merck, Darmstadt, Germany) was added to each Eppendorf tube containing a D-Squame. Samples were mixed by vortexing for 30 minutes on ice and then subjected to ultrasound sonication (Hielscher, Ultrasound Technology, Milan Italy). Extracts were centrifuged (10 minutes at 10,000 g), and supernatant aliquots were frozen at −80° C. until analysis.

### Determination of Lipid Hydroperoxide Levels

Lipid peroxide (LPO) levels in sebum were determined by spectrophotometric ferric-xylenol orange peroxide assay (Xylenol Orange Assay; described in Gay and Gebicki, (2002) “Perchloric acid enhances sensitivity and reproducibility of the ferric-xylenol orange peroxide assay” Anal Biochem 304(1):42-6). LPO results were expressed as H₂O₂ μmol/mg sebum, measuring the optical density at 560 nm.

### Statistical Analysis

Statistical significance was assessed using Student's t-test and ANOVA method. The minimal level of significance was P<0.05.

### Results

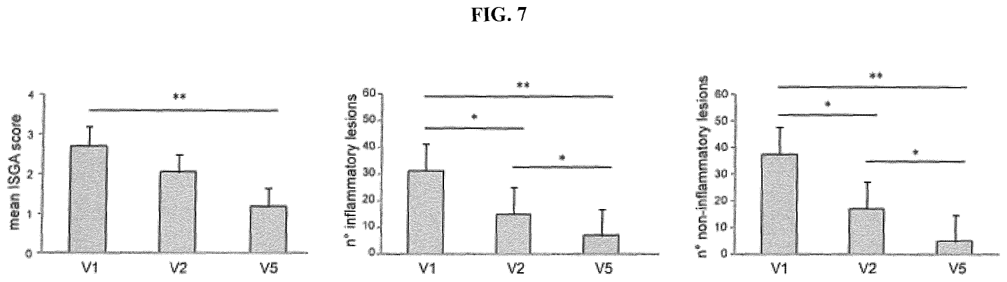

To determine whether administering a PPARγ modulator could effectively treat acne, acne patients were treated daily with 1% NAC-GED0507 gel for 12 weeks (84 days) and monitored for acne severity using the ISGA score. The ISGA score is measured on a 6-point ordinal scale, where 0=Clear and 5=Very Severe. Treatment with NAC-GED0507 gel for 12 weeks resulted in a significant reduction of acne symptoms as demonstrated by a reduction of ISGA score (FIG. 7). Treatment resulted in an observable reduction in the number of inflammatory and non-inflammatory global lesions in patients throughout the course of the trial (FIG. 7). This result demonstrates that daily topical administration of the PPARγ modulator NAC-GED0507 reduced acne severity in acne patients.

### Example 8: Topical Treatment with NAC-GED0507 Increases PPARγ Levels and Inhibits mTOR Pathway Activation

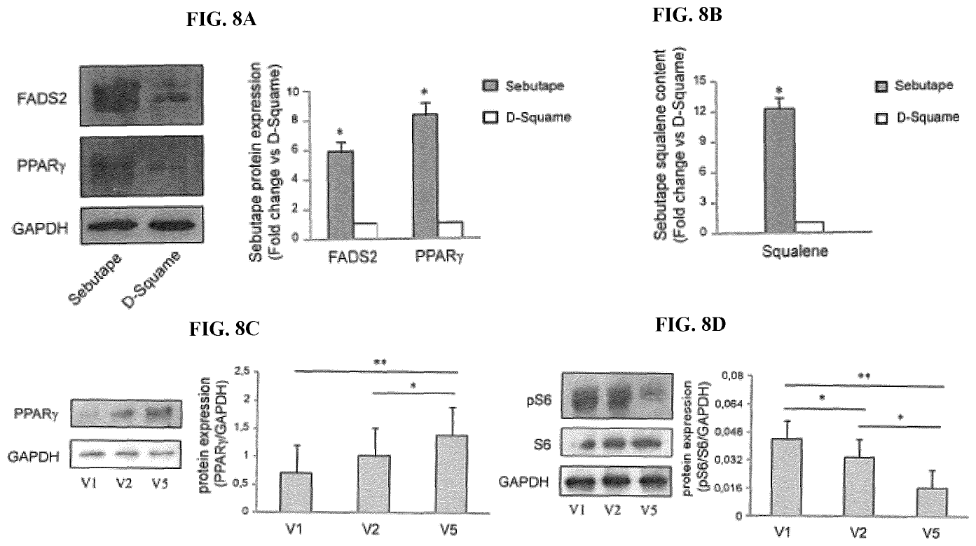

Levels of FADS2 (skin expression of which is limited to sebaceous glands) and PPARγ protein and the sebum-specific component squalene were measured in extracts collected using Sebutape and D-Squame. Western blot analysis demonstrated that levels of FADS2 and PPARγ were approximately 6 to 8 times higher in extracts collected using Sebutape as compared to D-Squame (FIG. 8A). GC-MS analysis demonstrated that extracts from Sebutape contained about 12 times more squalene than extracts from D-Squame (FIG. 8B). These results demonstrate that sebum collected using Sebutape contains significantly higher amounts of squalene, FADS2, and PPARγ, as compared to stratum corneum extracts collected using D-Squame.

Sebaceous glands of acne patients express decreased levels of PPARγ and increased levels of mTOR and pS6 as compared to sebaceous glands of healthy control subjects (Dozsa A et al., 2016; Monfrecola, 2015). To determine whether PPARγ modulation by NAC-GED0507 reduces the activation of Akt/mTOR signaling and ameliorates clinical signs of acne, protein expression levels of PPARγ and pS6 were measured in sebum from acne subjects, before and after topical application of 1% NAC-GED0507 gel for 84 days. Topical application of NAC-GED0507 resulted in a significant increase in PPARγ protein expression over the course of treatment (FIG. 8C). Topical application of NAC-GED0507 also resulted in a significant decrease in the expression of pS6 over the course of treatment (FIG. 8D). These results demonstrate that topical administration of NAC-GED0507 induced PPARγ protein expression in sebocytes of acne patients along with a significant decrease in Akt/mTOR signaling. These results also demonstrate that topical application of 1% NAC-GED0507 gel modified sebaceous gland differentiation state and metabolism, which is in line with in vitro results.

### Example 9: Topical Treatment with NAC-GED0507 Reduces the Inflammatory Process

To determine whether PPARγ modulation affects inflammatory cytokine production in acne patients, levels of the cytokine IL-1 alpha (IL-1α) were measured in stratum corneum extracts collected from lesional areas of skin and in sebum extracts from the foreheads of acne patients at various time points before and during the course of topical treatment with 1% NAC-GED0507 gel. Patients were treated daily for 84 days, as described above. Stratum corneum extracts were collected using D-Squame and sebum extracts were collected using Sebutape.

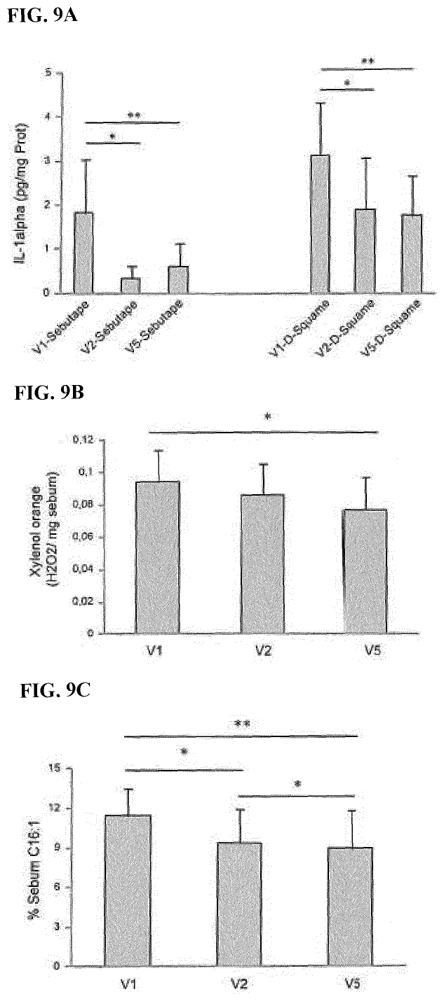

Stratum corneum levels of IL-1α at V2 and V5 were significantly decreased in comparison to levels at V1 in lesional areas (FIG. 9A). A slight decrease in stratum corneum IL-1α levels was observed at V5 as compared to V2. Similarly, sebum levels of IL-1α at V2 and V5 were significantly decreased in comparison to levels at V1 (FIG. 9A). The level of lipid peroxidation in sebum also decreased significantly during the course of administering NAC-GED0507 gel (FIG. 9B). Analysis of this data also demonstrated a significant increase in the ratio of PPARγ to IL-1α expression and the ratio of PPARγ expression to amount of lipid peroxidation. A significant increase in both of these ratios was observed, indicating a relationship between PPARγ expression and inflammation. These results demonstrate that topically administering NAC-GED0507 effectively counteracted inflammation in acne patients, particularly in sebocytes.

### Example 10: Topical Treatment with NAC-GED0507 Reduces the Percentage of Sapienic Acid in Acne Patient Sebum

Sapienic acid (C16:1 Δ6) is the most abundant mono-unsaturated fatty acid found in sebum, and it is presumed to play a role in acne pathogenesis (Ge L et al., JID, 2003). Decreases in the amount of sapienic acid in sebum from acne patients are correlated with the clinical improvement of acne (Smith R N, J Dermatol Sci, 2008). In order to determine whether topical treatment with the PPARγ modulator NAC-GED0507 affects sapienic acid sebum content, the amount of sapienic acid in sebum from acne patients was measured by GC-MS. Sapienic acid sebum content significantly decreased over the course of 84 days of daily topical administration of NAC-GED0507 gel (FIG. 9C). This result demonstrates that topical administration of NAC-GED0507 both reduced acne severity in patients and decreased enzymatic desaturase activity.

Overall, these results demonstrate that in vivo topical application of 1% NAC-GED0507 gel significantly ameliorated clinical manifestations of acne in patients. Topical application of 1% NAC-GED0507 gel also resulted in the induction of sebocyte differentiation marker expression and decreased expression of inflammatory molecules.

More generally, these results indicate that degree of sebocyte differentiation determines sebocyte responsiveness to insulin-stimulated lipidogenesis and correlates with likelihood of acne development. Additionally, these results indicate that the PPARγ modulator NAC-GED0507 is effective in inducing sebocyte differentiation and countering cellular responses associated with acne pathology, suggesting that administering NAC-GED0507 is effective for treating and preventing acne and related diseases, conditions, and disorders associated with alterations in sebocyte differentiation.

Unless defined otherwise, all technical and scientific terms used herein have the same meaning as commonly understood by one of ordinary skill in the art to which this invention belongs.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise. Further, unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present invention remain operable. Moreover, two or more steps or actions may be conducted simultaneously.

At various places in the present specification, variables are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

What is claimed is:

1. A method of preventing acne in a patient, the method comprising:
administering topically to the patient a composition effective to induce sebocyte differentiation,
wherein the composition comprises a therapeutically effective amount of a PPARγ modulator; and a pharmaceutically acceptable excipient.

2. A method of preventing acne in a patient, the method comprising:
administering topically to the patient a composition effective to reduce insulin-induced lipoxygenase (LOX) activity,
wherein the composition comprises a therapeutically effective amount of a PPARγ modulator; and a pharmaceutically acceptable excipient.

3. The method of claim 1 or 2, wherein the patient displays no signs or symptoms of acne.

4. A method of preventing a disease, condition, or disorder characterized by alteration of sebocyte differentiation in a patient, the method comprising:
administering topically to the patient a composition effective to induce sebocyte differentiation,
wherein the composition comprises a therapeutically effective amount of a PPARγ modulator; and a pharmaceutically acceptable excipient.

5. The method of any one of claims 1-4, wherein the method restores the physiological composition of secreted sebum to a level of mono-unsaturated fatty acids as found in patients without acne.

6. The method of claim 5, wherein the mono-unsaturated fatty acids are C16:1 mono-unsaturated fatty acids.

7. The method of any one of claims 1-6, wherein the method restores the physiological composition of secreted sebum to a level of diacylglycerides as found in patients without acne.

8. The method of any one of claims 4-7, wherein the disease, condition, or disorder is selected from one or more of acne, sebaceous hyperplasia, and sebaceous adenitis.

9. The method of any one of claims 1-8, wherein the PPARγ modulator is N-acetyl-(S)-3-(4′-aminophenyl)-2-methoxypropionic acid, or a pharmaceutically acceptable salt thereof.

10. The method of any one of claims 1-9, wherein the patient is experiencing puberty.

11. The method of any one of claims 1-10, wherein the patient has eaten or is eating an insulinotropic diet.

12. The method of any one of claims 1-11, wherein the composition is in the form of a gel.

13. The method of any one of claims 1-11, wherein the composition is in the form of an aerosol foam.
